(19) United States
(12) Patent Application Publication
(10) Pub. No.: US 2011/0190221 A1
(43) Pub. Date: Aug. 4, 2011

(54) MODULATION AND REPLETION/ENHANCEMENT OF THE COMPLEMENT SYSTEM FOR TREATMENT OF TRAUMA

(75) Inventors: Cedric Francois, Louisville, KY (US); Pascal Deschatelets, Louisville, KY (US); Paul Olson, Louisville, MO (US)

(73) Assignee: APELLIS AG, Louisville, KY (US)

(21) Appl. No.: 12/935,251

(22) PCT Filed: Mar. 30, 2009
(86) PCT No.: PCT/US09/38807

§ 371 Date: Apr. 14, 2011

Related U.S. Application Data
(60) Provisional application No. 61/040,637, filed on Mar. 28, 2008.
(60) Provisional application No. 61/040,634, filed on Mar. 28, 2008.
(60) Provisional application No. 61/040,635, filed on Mar. 28, 2008.

(51) Int. Cl. A61K 38/14; A61K 38/10; A61P 43/00

(52) U.S. Cl. 514/20.9; 514/21.5

(57) ABSTRACT

The present invention features the use of selected complement activation inhibitor(s) for treating an individual who has suffered a severe injury. The complement activation inhibitor acts at or above the level of C3 activation and does not significantly deplete or irreversibly inhibit complement activation. Also provided are compositions and methods for repleting and/or enhancing complement activation capacity in a subject who has suffered a traumatic injury.

### RELATED APPLICATION INFORMATION

This application claims priority to U.S. provisional applications U.S. Ser. No. 61/040,634, U.S. Ser. No. 61/040,635 and U.S. Ser. No. 61/040,637 all filed on Mar. 28, 2008 the contents of each of which are incorporated herein by reference.

### BACKGROUND OF THE INVENTION

Trauma patient usually refers to someone who has suffered serious physical injury such as open wounds, blunt injury, and major burns, potentially resulting in secondary complications such as shock, respiratory failure, sepsis, and death. Trauma patients often require specialized care, such as surgery and sometimes blood transfusion, within the so-called golden hour of emergency medicine, the first sixty minutes after trauma occurs. This is not a strict time limit, but recognizes that many deaths that could have been prevented by appropriate care occur a relatively short time after injury.

Traumatic injury is a worldwide problem affecting individuals of all ages and socioeconomic backgrounds. Its causes are equally diverse, ranging from motor vehicle crashes (41% of cases) to burns and falls (27% of cases), firearm-related events (6% of cases) and other interpersonal violence (6% of cases) to motor vehicle crashes (41% of cases), burns and falls (27% of cases) (U.S. data, National Trauma Data Bank Report 2006, version 6.0) An estimated 5 million people died as a result of injury in 2000, representing approximately 9% of worldwide deaths and an estimated 12-16% of the global disease burden. In the U.S. trauma is the fourth leading cause of death overall, accounting for over 160,000 deaths, and the leading cause of death among those aged 1-44 years. At least 1.4 million Americans experience a traumatic brain injury every year, resulting in 235,000 hospitalizations, 50,000 deaths, and 80,000 to 90,000 individuals suffering from permanent impairment. Burns result in approximately 100,000 hospitalizations and 5,000 deaths annually in the U.S. alone.

The social and economic burdens of traumatic injury are immense. In 2004, over $117 billion, or 10% of total medical expenditures, was spent on injury-related medical care in the U.S. (Centers for Disease Control and Prevention. MMWR Morb Mortal Wkly Rep., 53(1):1-4, 2004). The actual economic cost of trauma is much larger, in part because it affects individuals in a relatively younger segment of the population as compared with other leading causes of death and disability, resulting in significant lost productivity and work years as well as long-term care needs.

The clinical conditions associated with severe trauma include hemorrhagic shock and resuscitation, ischemia-reperfusion injury, acute respiratory distress syndrome (ARDS), thermal injury, inhalation injury, sepsis, and traumatic brain injury. Mortality rates for the injured vary significantly depending on injury severity, presence of shock or central nervous system (CNS) injury, physiologic reserve, and availability of appropriate care Immediate and early (within 24 hours) trauma deaths are due largely to primary central nervous system injury (˜40-50% of fatalities) and significant blood loss (˜30-40% of fatalities). During subsequent hospitalization, death from causes not directly related to specific injuries becomes more common. Systemic inflammatory response syndrome (SIRS), multi-organ failure (MOF), secondary brain damage, and infection are the major causes of death in the hospitalized trauma patient after the first 24-48 hours. CNS and respiratory system dysfunction are major contributors to early lethality after trauma. Secondary brain injuries and impairment of the hepatic, renal, gastrointestinal, hematopoietic, and cardiovascular systems typically manifest somewhat later. Post-traumatic ARDS and respiratory failure can occur early after injury in association with hemorrhagic shock or later in association with multiple organ system injury and pneumonia. Mortality due to MOF increases dramatically with the number of organ systems affected. With appropriate therapy, mortality is usually under 5% with single organ failure, increasing to 90% or more when at least four organ systems fail.

In the developed world, civilian trauma care provision is integrated from the level of field emergency service providers through outlying hospitals to specialized trauma care centers to which severely injured patients may be transported following initial stabilization. Initial prehospital care focuses on maintaining adequate airway patency, breathing, and circulation. Acute management of traumatic hemorrhage can be divided into the resuscitative, operative, and critical care phases, geared towards control of bleeding, prevention of coagulopathy, and hemodynamic stabilization. Injury-specific measures include wound care, mechanical ventilation cases of severe chest trauma or pulmonary dysfunction, surgery for fractures, repair of vascular injuries, and removal of penetrating objects. Management of severe traumatic CNS injury according to established guidelines has been shown to improve survival and functional outcomes. In the hospital setting, attention is paid to monitoring and correcting raised intracranial pressure. Thus much most post-trauma care is surgical and supportive in nature. There is a need in the art for new approaches to treating trauma patients.

### SUMMARY OF THE INVENTION

The invention provides compositions and methods for treating an individual who has suffered a traumatic injury. Certain of the methods comprise administering a complement activation inhibitor that acts at or above the level of C3 activation wherein the agent is administered during the immediate post-trauma period.

In one aspect, the invention provides method of treating an individual who has suffered a traumatic injury comprising administering a complement activation inhibitor to the individual within 12 hours after the injury, wherein the complement inhibitor (i) acts at or above the level of C3 activation; and (ii) does not cause significant depletion or irreversible inactivation of an unactivated complement component. In some embodiments the complement activation inhibitor acts on a complement component selected from the group consisting of: C1, C2, C3, C4, factor B, factor D, or an active fragment of one or more of the foregoing. In some embodiments the complement inhibitor is administered within 4 hours after the injury. In some embodiments the complement inhibitor has an average half-life of 24 hours or less in humans under normal conditions. In some embodiments the complement inhibitor has an average half-life of 12 hours or less in humans under normal conditions. In some embodiments the complement inhibitor is administered in an amount such that, by 24 hours after the injury, the individual's potential alternative pathway activity, potential classical pathway activity, or both, is/are significantly preserved. In some embodiments the complement inhibitor is administered in an amount such that, by 48 hours after the injury, the individual's potential alternative pathway activity, potential classical pathway activity, or both the individual's potential classical pathway activity and the individual's potential alternative pathway activity is/are significantly preserved. In some embodiments the complement inhibitor is administered in an amount such that, by 72 hours after the injury, the individual's potential alternative pathway activity, potential classical pathway activity, or both the individual's potential classical pathway activity and the individual's potential alternative pathway activity is/are significantly preserved. In some embodiments the complement activation inhibitor is administered intravenously. In some embodiments the complement activation inhibitor is administered in the field. In some embodiments the complement activation inhibitor is administered in a fixed dose format. In some embodiments the individual does not have a condition, other than the injury, for which complement inhibitor therapy (e.g., systemic complement inhibition) is indicated. In some embodiments the complement inhibitor comprises a soluble complement receptor or a complement inhibiting altered version thereof. In some embodiments the complement inhibitor comprises soluble CR1 or an altered version thereof that binds to C1. In some embodiments the complement inhibitor comprises an antibody or antibody fragment that binds to C1. In some embodiments the complement inhibitor inhibits a C3 convertase.

In some embodiments the complement inhibitor binds to C3 and inhibits its activation. In some embodiments the complement inhibitor comprises a compstatin analog. In some embodiments the complement activation inhibitor comprises a compstatin analog having a sequence selected from the group consisting of: SEQ ID NOs: 14, 21, 28, 29, 30, 32, 33, 34, or 36. In some embodiments the complement activation inhibitor comprises a VCCP or VCIP or complement inhibiting altered version thereof. In some embodiments the complement activation inhibitor comprises a mammalian complement regulatory protein or a complement inhibiting altered version thereof. In some embodiments the complement activation inhibitor comprises a mammalian complement control protein or a complement inhibiting fragment or altered version thereof, wherein the mammalian complement control protein is decay accelerating factor (DAF). In some embodiments the complement activation inhibitor comprises a mammalian complement regulatory protein or a complement inhibiting fragment or altered version thereof, wherein the mammalian complement control protein is membrane cofactor protein (MCP). In some embodiments the complement inhibitor comprises a chimeric protein comprising portions of two different mammalian complement regulatory proteins. In some embodiments the complement activation inhibitor comprises at least a portion of DAF and at least a portion of MCP or altered version thereof. In some embodiments the complement activation inhibitor comprises a polypeptide whose sequence is at least 80% identical to the sequence of CAB-2. In some embodiments the complement activation inhibitor binds to factor B and inhibits its activation. In some embodiments the complement activation inhibitor is an antibody or antibody fragment that binds to factor B and inhibits its activation and is administered within less than 1 hour after trauma. In some embodiments the complement activation inhibitor binds to factor D and inhibits its activation. In some embodiments, the method further comprises administering an effective amount of an additional agent, wherein the additional agent stabilizes a second complement component, which in some embodiments is C5. In some embodiments, the method further comprises administering an effective amount of an agent that inhibits cleavage of C5. In some embodiments, the agent inhibits C3-independent cleavage of C5. In some embodiments, the method further comprises administering to the individual an unactivated complement component. In some embodiments, if the individual receives blood, blood product, or cells, the complement component is in addition to any unactivated complement component that is naturally present in blood, blood product, or cells administered to the individual. In some embodiments the complement component is administered at least 4 hours after the injury. In some embodiments the complement component is administered at least 24 hours after the injury. In some embodiments the complement component is administered between 24 hours and 2 weeks after the injury. In some embodiments the complement component is administered at least once within 2 weeks after the injury and at least once more than 2 weeks after the injury. In some embodiments the complement component is an at least partially purified or recombinantly produced protein. In some embodiments the complement component is a component of the classical pathway. In some embodiments n the complement component is a component of the alternative pathway. In some embodiments the complement component is a component of the alternative and classical pathways. In some embodiments the complement component is a component of the mannose binding lectin pathway. In some embodiments the complement component is selected from the group consisting of: C1, C2, C3, C4, C5, C6, C7, C8, C9, and properdin. In some embodiments the complement component is complexed with an agent that inhibits its activation but does not irreversibly inactivate it. In some embodiments the complement component is C3, and wherein the C3 is complexed with a compstatin analog. In some embodiments the complement component is C3, and wherein the C3 is complexed with a compstatin analog selected from SEQ ID NOs: 14, 21, 28, 29, 30, 32, 33, 34, or 36.

In another aspect, the invention provides a method of treating an individual who has suffered a traumatic injury comprising administering a complement activation inhibitor to the individual in the immediate post-trauma period in an amount effective to reduce trauma-induced complement depletion by at least 50%, wherein the complement inhibitor does not itself cause significant depletion or irreversible inactivation of an unactivated complement component, and wherein the complement inhibitor is administered in an amount such that at least 90% of the complement inhibitor administered is gone from the blood within 1 week after the injury. In some embodiments at least 90% of the complement inhibitor administered is gone from the body or from the blood or plasma within 1 week after injury. In some embodiments the individual does not have a condition, other than the injury, for which complement inhibition, e.g., systemic complement inhibition, is indicated. In some embodiments the complement activation inhibitor is administered within 1, 2, 4, 6, or 12 hours after injury.

In another aspect, the invention provides a composition comprising: (a) a first agent that inhibits depletion of a complement component at or above the level of C3 activation; and (b) a second agent that stabilizes C5. In some embodiments the first agent is an aptamer, peptide, antibody, or antibody fragment that binds to the complement component. In some embodiments the second agent inhibits C3-independent cleavage of C5. In some embodiments the second agent is an aptamer, peptide, antibody, or antibody fragment that binds to C5. In some embodiments the composition further comprises a fluid suitable for intravenous (IV) administration to a trauma patient. In some embodiments the fluid is selected from the group consisting of: physiologically acceptable saline, Ringer's lactate, 5% dextrose in water, blood or a blood product, colloid-containing volume expander, and wherein the fluid optionally contains one or more additional constituents suitable for IV administration. The invention further provides an IV solution bag containing any of the afore-mentioned inventive compositions. The invention further provides a method of treating an individual who has suffered a traumatic injury comprising administering any of the afore-mentioned compositions to the individual. In some embodiments the composition is administered within 1 hour or less after the injury. In some embodiments the composition is administered within 12 hours or less after the injury.

In another aspect, the invention provides a composition suitable for administration to a trauma patient, the composition comprising: (a) a complement component; and (b) a complement stabilizing agent that stabilizes the complement component or a cleavage product thereof or in active form or in inactive but activatable form, wherein if the composition comprises blood, blood product, or cells, the complement component or complement stabilizing agent differs in amount or identity from such complement component(s) or complement stabilizing agent(s) as are naturally present as a constituent of said blood, blood product, or cells. In some embodiments the complement stabilizing agent is an allosteric regulator. In some embodiments the complement stabilizing agent is an antibody fragment, aptamer, peptide, or small molecule ligand that binds to the complement component. In some embodiments the complement component is a component of the classical pathway. In some embodiments the complement component is a component of the alternative pathway. In some embodiments the complement component is a component of the alternative and classical pathways. In some embodiments the complement component is a component of the mannose binding lectin pathway. In some embodiments the complement stabilizing agent comprises at least a portion of a complement regulatory protein. In some embodiments the complement stabilizing agent is properdin. In some embodiments the complement component is inactive but is activatable following dissociation from the complement stabilizing agent. In some embodiments the agent stabilizes the complement component in inactive but activatable form. In some embodiments the agent specifically binds to the complement component. In some embodiments the complement component and agent are complexed with one another in a complex that has a half-life of at least 1 hour in vitro. In some embodiments the complement component or cleavage product is selected from the group consisting of: C1, C3, C3b, and C5. In some embodiments the complement component is C3 and the complement stabilizing agent is an aptamer, peptide, antibody, or antibody fragment that binds to C3 or C3b. In some embodiments the complement component is C3 and the complement stabilizing agent is a compstatin analog. In some embodiments the complement component is C3 and the complement stabilizing agent is a compstatin analog selected from the group consisting of: SEQ ID NOs: 14, 21, 28, 29, 30, 32, 33, 34, or 36. In some embodiments the composition further comprises an additional complement component or complement stabilizing agent. In some embodiments the composition further comprises a fluid suitable for IV administration to a trauma patient. In some embodiments the IV fluid is selected from the group consisting of: saline, Ringer's lactate, 5% dextrose in water, blood or a blood product, colloid-containing volume expander, and wherein the fluid optionally contains one or more additional constituents suitable for IV administration. The invention further provides an IV solution bag containing any of the afore-mentioned compositions. The invention further provides methods of treating an individual who has suffered a traumatic injury comprising administering any of the afore-mentioned compositions to the individual. In some embodiments the composition is administered between 4 hours and 4 days after the injury. In some embodiments the composition is administered within 12 hours or less after the injury. In some embodiments the composition is administered within 24 hours or less after the injury. In some embodiments the composition is administered more than 24 hours after the injury.

In another aspect, the invention provides composition comprising: (a) a complement component; and (b) a fluid suitable for IV administration to an individual who has suffered a traumatic injury, wherein if the composition comprises blood, blood product, or cells, the complement component differs in amount or identity from such complement component(s) as are naturally present as a constituent of said blood, blood product, cells, or tissue. In some embodiments the complement component is a component of the classical pathway. In some embodiments the complement component is a component of the alternative pathway. In some embodiments the complement component is C3. In some embodiments the complement component is factor B or factor D. In some embodiments the complement component is properdin. In some embodiments the complement component is a component of the alternative and classical pathways. In some embodiments the complement component is a component of the mannose binding lectin pathway. In some embodiments the IV fluid is selected from the group consisting of: saline, Ringer's lactate, 5% dextrose in water, blood or a blood product, colloid-containing volume expanders, and wherein said fluid optionally contains one or more additional constituents suitable for IV administration. The invention further provides methods of treating an individual who has suffered a traumatic injury comprising administering any of the afore-mentioned compositions to the individual. In some embodiments the composition is administered within 24 hours or less after the injury, with the proviso that the complement component is not properdin. In some embodiments the composition is administered more than 24 hours after the injury.

In another aspect, the invention provides a pharmaceutical composition comprising: (i) properdin; and (ii) a pharmaceutically acceptable carrier. The invention also provides a method of treating an individual who has suffered a traumatic injury comprising administering the afore-mentioned composition to the individual, e.g., more than 24 hours after the injury.

The invention further provides a humanized antibody that binds to a mammalian complement regulatory protein. In some embodiments the complement regulatory protein is selected from the group consisting of: CFH, CFI, and C4bp. The invention further provides a pharmaceutical composition comprising the antibody and a pharmaceutically acceptable carrier. The invention further provides a method of treating an individual who has suffered a traumatic injury comprising administering the composition to the individual. In some embodiments the composition is administered at least 4 hours after the injury. In some embodiments the composition is administered at least 48 hours after the injury.

In one aspect, the invention provides a humanized antibody or antibody fragment that binds to complement factor H(CFH). In some embodiments the antibody selectively binds to CFH having tyrosine at position 402 relative to CFH having histidine at position 402. The invention further provides a pharmaceutical composition comprising the antibody and a pharmaceutically acceptable carrier. The invention further provides a method of treating an individual who has suffered a traumatic injury comprising administering the composition comprising the antibody or antibody fragment to the individual. In some embodiments the composition is administered more than 4 after the injury. In some embodiments the composition is administered at least 48 after the injury and within 2 weeks after the injury. In some embodiments the composition is administered at least 24 hours after the injury and within 72 hours after the injury.

The invention provides a method of treating an individual who has suffered a traumatic injury comprising administering an agent that enhances the individual's potential complement activity, wherein the agent is administered at an appropriate time point after the injury so as to decrease the likelihood that the individual will experience a poor outcome. In some embodiments the time point is at least 4 hours after the injury and within 4 days after the injury.

The invention provides a method of treating an individual who has suffered a traumatic injury comprising administering a composition comprising a compstatin analog to the individual within the first 24 hours after occurrence of the injury. In some embodiments the compstatin analog has a greater activity than compstatin. In some embodiments the compstatin analog has a sequence selected from the group consisting of: SEQ ID NOs: 14, 21, 28, 29, 30, 32, 33, 34, and 36. In some embodiments the method further comprises administering a composition comprising a complement component to the individual within 4 hours and 4 days after occurrence of the injury.

In another aspect, the invention provides a pharmaceutical composition comprising a CFH polypeptide, wherein the pharmaceutical composition is suitable for IV administration. In some embodiments the CFH polypeptide has a histidine at position 402. The invention further provides a method of treating an individual who has suffered a traumatic injury comprising administering a CFH polypeptide to the individual. In some embodiments the polypeptide is a CFH polypeptide having histidine at position 402.

While the invention is described with reference to traumatic injury, the inventive compositions and methods also find use in patients that have undergone significant surgery, and such embodiments are within the scope of the invention. In some embodiments a significant surgery is a cardiac surgery, thoracic surgery, abdominal surgery, orthopedic surgery, or neurosurgery. In some embodiments a significant surgery last for longer than 4 hours and/or requires blood transfusion and/or requires ICU care for at least 1 day.

All articles, books, patent applications, patents, and other publications, mentioned in this application are incorporated herein by reference. Current Protocols in Molecular Biology, Current Protocols in Immunology, Current Protocols in Protein Science, and Current Protocols in Cell Biology, all John Wiley & Sons, N.Y., edition as of 2008; Sambrook, Russell, and Sambrook, Molecular Cloning: A Laboratory Manual, 3rd ed., Cold Spring Harbor Laboratory Press, Cold Spring Harbor, 2001; Kuby Immunology, 5th ed., Goldsby, R. A., Kindt, T. J., and Osborne, B. (eds.), W.H. Freeman, 2006, Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 9th edition (December 2003); Goldman & Ausiello, Cecil Textbook of Medicine, 22nd ed., W.B. Saunders, 2003. Fundamental Immunology, Lippincott Williams & Wilkins; 5th ed., 2003 Applicants′ provisional applications U.S. Ser. No. 61/040,634; 61/040,635, and 61/040,637, filed Mar. 28, 2008, are incorporated herein by reference. Art-accepted abbreviations for amino acids, genes, and other terms are used herein.

### DEFINITIONS

As used herein, a first entity (e.g., an inhibitor) acts “directly” on a second entity (e.g., a molecule to be inhibited) if the first entity physically interacts with the second entity and thereby affects a property or activity of the second entity. A first entity acts “indirectly” on a second entity if the first entity affects a property or activity of the second entity by a process that does not involve physical interaction of the first and second entitities.

“Approximately” or “about” in reference to a number generally include numbers that fall within ±10%, in some embodiments ±5%, in some embodiments ±1%, in some embodiments ±0.5% of the number unless otherwise stated or otherwise evident from the context (except where such number would impermissibly exceed 100% of a possible value).

A “complement component” or “complement protein” is a molecule or molecular complex that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, CSa, etc.). Components of the alternative pathway include, e.g., factor B, factor D, and properdin. Components of the lectin pathway include, e.g., MBL2, MASP-1, MASP-2, MASP-3, and sMAP/Map19. For purposes of this disclosure, the term “complement component” is not intended to include complement receptors or complement regulatory proteins. The term “complement component” is also not intended to include those molecules and molecular structures that serve as “triggers” for complement activation, e.g., antigen-antibody complexes, foreign structures found on microbial or articifial surfaces, etc.

A “complement regulatory protein” is a protein involved in regulating complement activity. Mammalian complement regulatory proteins include membrane cofactor protein (MCP; CD46), decay accelerating factor (DAF; CD55); complement factor H(CFH), complement factor I, and CD59.

A “complement control protein” is a complement regulatory protein comprising multiple short consensus repeat (SCR) modules as described below. Mammalian complement control proteins include MCP, DAF, and CFH.

A “complement-like protein” is a protein that has significant sequence identity to a complement protein or a complement control protein over at least 20% of its length and/or specifically competes with the complement protein or complement control protein for binding to its target, e.g., has an affinity at least 10% as great. The genes encoding such proteins may be found in close proximity to genes encoding the complement protein or complement control protein having a similar sequence. For example, the CFH gene cluster contains numerous CFH-like genes (e.g., CFHR1, CFHR1, CFHR3, CFHR4, and CFHR5). Aspects of the invention that relate to CFH include embodiments that relate to one or more CFH-like genes and embodiments that relate solely to CFH.

“Complement-related protein” refers collectively to complement components, complement regulatory proteins, complement-like proteins and cell-bound receptors for soluble complement components, e.g., C5a receptor (C5aR), C3a receptor (C3aR), Complement Receptor 1 (CR1), Complement Receptor 2 (CR2), Complement Receptor 3 (CR3), etc.; Where this disclosure refers to complement-related proteins in general, it is understood that the invention encompasses embodiments that relate specifically to complement components, complement regulatory proteins, complement-like proteins, complement receptors, and any combination thereof.

The term “gene” is used as understood in the art. In mose cases a gene comprises a nucleic acid sequence that encodes a polypeptide and can also include intron sequences and other regions that are transcribed into RNA, regulatory sequences (e.g., promoters, enhancers), etc. It will be appreciated that a “gene” can encode a functional RNA such as a microRNA, tRNA, etc., rather than a polypeptide.

“Identity” refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. By gap is meant a portion of a sequence that is not occupied by a residue. For example, the sequence A K L - - - SIG (SEQ ID NO: 1) contains a gap of three residues. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between a sequence of interest and sequences in any of a variety of public databases. The algorithm of Karlin and Altschul (Karlin and Altschul, Proc. Natl. Acad. Sci. USA 87 22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. See the Web site having URL www.ncbi.nlm.nih.gov for these programs. In a specific embodiment, percent identity of a sequence of interest and a second sequence is calculated using BLAST2 with default parameters.

“Normal” is used herein consistently with usage in the art to refer to a subject who is in good health and does not suffer from a disease or condition recognized as being associated with abnormality of the complement system, and whose complement system is not at that time being affected by trauma, surgery, or other events that would be expected to significantly affect the concentration, absolute level, or activation state of complement components in the subject. For example, the subject has not suffered such event, or the subject's complement system has returned essentially to the state it was in prior to such event. A “normal” value used in the invention can be an average or median value or a value falling within a reference range defined as the set of values that 95% of the normal population falls within, or two standard deviations from the mean. In some embodiments, a “normal value” is a value between 50% and 150% of the average value found in a given population (and/or within a subpopulation similar in terms of weight, age, and other demographic factors). In some embodiments, a “normal value” is a value between 75% and 125% of the average value found in a given population (and/or within a subpopulation similar in terms of weight, age, and other demographic factors). In some embodiments, a “normal value” is a value between 90% and 110% of the average value found in a given population (and/or within a subpopulation similar in terms of weight, age, and other demographic factors). Normal values and average values for parameters such as level of complement components present in the blood or plasma, level of complement activation capacity, etc., are available and may be used in the invention.

“Plurality” means more than one.

“Polypeptide”, as used herein, refers to a polymer of amino acids, optionally including one or more amino acid analogs. A protein is a molecule composed of one or more polypeptides. A peptide is a relatively short polypeptide, typically between about 2 and 60 amino acids in length, e.g., between 8 and 40 amino acids in length. The terms “protein”, “polypeptide”, and “peptide” may be used interchangeably. Polypeptides used herein may contain amino acids such as those that are naturally found in proteins, amino acids that are not naturally found in proteins, and/or amino acid analogs that are not amino acids. As used herein, an “analog” of an amino acid may be a different amino acid that structurally resembles the amino acid or a compound other than an amino acid that structurally resembles the amino acid. A large number of art-recognized analogs of the 20 amino acids commonly found in proteins (the “standard” amino acids) are known. One or more of the amino acids in a polypeptide may be modified, for example, by the addition of a chemical entity such as a carbohydrate group, a phosphate group, a farnesyl group, an isofarnesyl group, a fatty acid group, a linker for conjugation, functionalization, or other modification, etc. Certain non-limiting suitable analogs and modifications are described in WO2004026328. The polypeptide may be acetylated, e.g., at the N-terminus and/or amidated, e.g., at the C-terminus. Polypeptides may, for example, be purified from natural sources, produced in vitro or in vivo in suitable expression systems using recombinant DNA technology in suitable expression systems (e.g., by recombinant host cells or in transgenic animals or plants), synthesized through chemical means such as conventional solid phase peptide synthesis and/or methods involving chemical ligation of synthesized peptides. Polypeptides may contain one or more non-naturally occurring amino acids or amino acid analogs or be otherwise modified. Activity of certain polypeptides is at least partly dependent on their glycosylation state. It may be desirable to produce such polypeptides in systems that provide for glycosylation similar or substantially identical to that found in mammals, e.g., humans. For example, mammalian expression systems (e.g., CHO cells), insect expression systems (e.g., Spodoptera frugiperda Sf9 cells), or modified lower eukaryotic expression systems (e.g., fungal expression systems), that provide for mammalian-like glycosylation can be used. See, e.g., U.S. Pub. Nos. 20060177898 and 20070184063.

“Significant sequence identity” as applied to an amino acid sequence means that the sequence is at least approximately 20%, 30%, 40%, 50%, 60%, 70%, 80%, 90%, or 95% identical to a reference sequence. In specific embodiments the sequence is at least approximately 70%, 80%, 85%, 90%, 95%, 98%, or 99% identical to a reference sequence. In specific embodiments at least 20%, 30%, 40%, 50%, 60%, 70%, 80%, 90%, or 95% of the nonidentical amino acids are conservatively replaced relative to the reference sequence. Conservative replacements may be defined in accordance with Stryer, L., Biochemistry, 3rd ed., 1988, according to which amino acids in the following groups possess similar features with respect to side chain properties such as charge, hydrophobicity, aromaticity, etc. (1) Aliphatic side chains: G, A, V, L, I; (2) Aromatic side chains: F, Y, W; (3) Sulfur-containing side chains: C, M; (4) Aliphatic hydroxyl side chains: S, T; (5) Basic side chains: K, R, H; (6) Acidic amino acids: D, E, N, Q; (7) Cyclic aliphatic side chain: P, which may be considered to fall within group (1). In another accepted classification, conservative substitutions occur within the following groups: (1) Non-polar: A, L, I, V, G, P, F, W, M; (2) Polar: S, T, C, Y, N, Q. (3) Basic: K, R, H; (4) Acidic: D, E. Amino acids with a small side chain (G, A, S, T, M) also form a group from among which conservative substitutions can be made. Other classification methods known in the art can be used. Furthermore, amino acid analogs and unnatural amino acids can be classified in accordance with these schemes.

A “subject” can be a human, non-human primate, dog, cat, rodent (e.g., mouse, rat), etc. Typically as used herein the term refers to an individual to whom a composition of the invention may be administered, e.g., an individual who has suffered traumatic injury. In embodiments of particular interest the subject is a human.

“Trauma” or “traumatic injury” as used herein refers to a severe physical injury due to a cause other than surgery.

An “altered version” of a particular polypeptide has one or more alterations (e.g., additions, substitutions, and/or deletions) with respect to that polypeptide, which polypeptide may be referred to as the “original polypeptide”. An altered version can be the same length as the original polypeptide or or may be shorter or longer. The term “altered version” encompasses “fragments”. A “fragment” is a continuous portion of a polypeptide that is shorter than the original polypeptide. In some embodiments the fragment comprises at least 30%, 40%, 50%, 60%, 70%, 80%, 90%, or 95% of the full length polypeptide. In certain embodiments of the invention an altered version is at least 50%, at least 60%, at least 70%, at least 80%, at least 90%, at least 95%, at least 97%, or at least 99% identical to the original polypeptide over a continuous portion of the altered version that comprises at least 50%, at least 60%, at least 70%, at least 80%, at least 90%, at least 95%, at least 97%, or at least 99%, or 100% of the length of the altered version or the length of the original polypeptide, (whichever is shorter). In a non-limiting embodiment an altered version has at least 80% identity to the original polypeptide over a continuous portion of the altered version that comprises between 90% and 100% of the altered version, e.g., over 100% of the length of the altered version or the length of the original polypeptide, (whichever is shorter). In another non-limiting embodiment an altered version has at least 90% identity to the original polypeptide over a continuous portion of the altered version that comprises between 90% and 100% of the altered version, e.g., over 100% of the length of the altered version or the length of the polypeptide, (whichever is shorter). In specific embodiments the sequence of an altered version polypeptide has N amino acid differences with respect to an original polypeptide, wherein N is any integer between 1 and 10. In other specific embodiments the sequence of an altered version polypeptide has N amino acid differences with respect to an original sequence, wherein N is any integer between 1 and 20. An amino acid “difference” refers to a substitution, insertion, or deletion of an amino acid. In some embodiments of the invention an altered version has significant sequence identity to the original polypeptide. In some embodiments of the invention an altered version polypeptide is one that has sufficient structural similarity to the original polypeptide so that when its 3-dimensional structure (either actual or predicted structure) is superimposed on the structure of the original polypeptide, the volume of overlap is at least 70%, preferably at least 80%, more preferably at least 90% of the total volume of the structure of the original polypeptide. A partial or complete 3-dimensional structure of the altered version may be determined by crystallizing the protein, which can be done using standard methods. Alternately, an NMR solution structure can be generated, also using standard methods. A modeling program such as MODELER (Sali, A. and Blundell, T L, J. Mol. Biol., 234, 779-815, 1993), or any other modeling program, can be used to generate a predicted structure. If a structure or predicted structure of a related polypeptide is available, the model can be based on that structure. The PROSPECT-PSPP suite of programs can be used (Guo, J T, et al., Nucleic Acids Res. 32(Web Server issue):W522-5, Jul. 1, 2004).

If an activity (e.g., a biochemical or biological activity) of an original polypeptide is also possessed by the altered version polypeptide, the altered version is said to be “active” with respect to that activity. An active altered version may be active with respect to one, more than one, or all known activities of the original polypeptide. An active altered version may have an activity that is at least 10%, at least 25%, at least 50%, at least 60%, at least 70%, at least 80%, or at least 90%, at least 100% of the activity of the original polypeptide, on a per molecule basis. Active altered versions may have increased activity relative to the original polypeptide. For example, the activity of the altered version may exceed that of the original polypeptide by a factor of 1.001 to 1000. In some embodiments an activity of an altered version is within a factor of 0.5 to 5 of that of the original polypeptide. A relevant activity in the case of some of the polypeptides of interest herein is ability to bind to a target molecule, e.g., a complement component. A relevant activity in the case of some of the polypeptides of interest herein is ability to inhibit cleavage of a complement component. A relevant activity in the case of some of the polypeptides of interest herein is ability to inhibit complement activation. A “complement inhibiting” altered version of a polypeptide is an altered version that is active with respect to complement inhibition. Complement inhibiting altered versions of complement inhibiting polypeptides described herein are of use in certain of the inventive methods. Altered versions of complement components are of use in certain of the inventive methods.

As used herein, “halo” refers to F, Cl, Br or I.

As used herein, “alkanoyl” refers to an optionally substituted straight or branched aliphatic acyclic residue having about 1 to 10 carbon atoms (and all combinations and subcombinaations of ranges and specific number of carbon atoms) therein, e.g., from about 1 to 7 carbon atoms. Alkanoyl groups include, but are not limited to, formyl, acetyl, propionyl, butyryl, isobutyryl, pentanoyl, isopentanoyl, 2-methyl-butyryl, 2,2-dimethoxypropionyl, hexanoyl, heptanoyl, octanoyl, and the like. “Lower alkanoyl” refers to an optionally substituted straight or branched aliphatic acyclic residue having about 1 to about 5 carbon atoms (and all combinations and subcombinaations of ranges and specific number of carbon atoms). Such groups include, but are not limited to, formyl, acetyl, propionyl, butyryl, isobutyryl, pentanoyl, isopentanoyl, etc.

As used herein, “aryl” refers to an optionally substituted, mono- or bicyclic aromatic ring system having from about 5 to about 14 carbon atoms (and all combinations and subcombinations of ranges and specific numbers of carbon atoms therein), with from about 6 to about 10 carbons being preferred. Non-limiting examples include, for example, phenyl and naphthyl.

As used herein, “aralkyl” refers to alkyl radicals bearing an aryl substituent and have from about 6 to about 22 carbon atoms (and all combinations and subcombinations of ranges and specific numbers of carbon atoms therein), with from about 6 to about 12 carbon atoms being preferred in certain embodiments. Aralkyl groups can be optionally substituted. Non-limiting examples include, for example, benzyl, naphthylmethyl, diphenylmethyl, triphenylmethyl, phenylethyl, and diphenylethyl.

As used herein, the terms “alkoxy” and “alkoxyl” refer to an optionally substituted alkyl-O— group wherein alkyl is as previously defined. Exemplary alkoxy and alkoxyl groups include methoxy, ethoxy, n-propoxy, i-propoxy, n-butoxy, and heptoxy.

As used herein, “carboxy” refers to a —C(═O)OH group.

As used herein, “alkoxycarbonyl” refers to a —C(═O)O-alkyl group, where alkyl is as previously defined.

As used herein, “aroyl” refers to a —C(═O)-aryl group, wherein aryl is as previously defined. Exemplary aroyl groups include benzoyl and naphthoyl.

Typically, substituted chemical moieties include one or more substituents that replace hydrogen. Exemplary substituents include, for example, halo, alkyl, cycloalkyl, aralkyl, aryl, sulfhydryl, hydroxyl (—OH), alkoxyl, cyano (—CN), carboxyl (—COOH), —C(═O)O-alkyl, aminocarbonyl (—C(═O)NH₂), —N-substituted aminocarbonyl (—C(═O)NHR″), CF₃, CF₂CF₃, and the like. In relation to the aforementioned substituents, each moiety R″ can be, independently, any of H, alkyl, cycloalkyl, aryl, or aralkyl, for example.

As used herein, “L-amino acid” refers to any of the naturally occurring levorotatory alpha-amino acids normally present in proteins or the alkyl esters of those alpha-amino acids. The term D-amino acid” refers to dextrorotatory alpha-amino acids. Unless specified otherwise, all amino acids referred to herein are L-amino acids.

As used herein, an “aromatic amino acid” is an amino acid that comprises at least one aromatic ring, e.g., it comprises an aryl group.

As used herein, an “aromatic amino acid analog” is an amino acid analog that comprises at least one aromatic ring, e.g., it comprises an aryl group.

### DETAILED DESCRIPTION OF CERTAIN EMBODIMENTS OF THE INVENTION

Overview

The present invention provides compositions and methods for treating an individual who has suffered a traumatic injury. Common causes of traumatic injury are motor vehicle accidents, falls, fires, interpersonal or self-inflicted violence (e.g., involving weapons such as guns or knives), occupational accidents, and combat-related events. In some embodiments of the invention the traumatic injury includes blunt trauma, penetrating trauma, crush injury, thermal injury, inhalation injury, and/or one or more fractures. In some embodiments of the invention the subject has suffered a single type of injury while in some embodiments the subject has suffered multiple types of injury.

A number of studies have found that complement activation occurs after severe injury and appears to correlate with injury severity, with increased complement activation being associated with worse outcomes. Several studies have suggested that complement activation contributes to many of the complications of severe trauma such as ischemia/reperfusion (I/R) injury, adult respiratory distress syndrome (ARDS), multi-organ dysfunction syndrome (MODS), multi-organ failure (MOF), secondary central nervous system (CNS) injury, and sepsis.

The present invention provides important insights for successful modulation of the complement system in trauma patients. In some embodiments, the invention provides methods for successful use of complement inhibitors in trauma patients. The methods of the invention preferably result in a statistically significant benefit among patients treated according to the invention relative to patients who do not receive such treatment but are otherwise matched in terms of factors such as injury severity, age, sex, and general quality of medical/surgical care they receive following trauma. For example, successful use of a complement inhibitor in trauma patients would be evidenced by a statistically significant reduction in poor outcome after traumatic injury. “Poor outcome” as used herein can refer to any of a number of undesirable complications, or events related to traumatic injury that are not directly and primarily attributable to acute blood loss (although blood loss and its consequences may be a contributing factor) or direct tissue damage from the injury and typically manifest (if at all) at least 24 hours following the injury, e.g., within 1, 2, 4, 6, 8, or 12 weeks after the injury. Poor outcomes include, e.g., (i) death; (ii) development of MOF; (iii) development of MODS; (iv) development of ARDS, (iv) sepsis; and (iv) any combination of (i)-(iv). A “favorable outcome” refers to an outcome other than a poor outcome. In some embodiments, a “favorable outcome” is an outcome other than a poor outcome under circumstances in which a poor outcome is considered more likely than not to occur. In some embodiments, a patient who recovers from trauma without experiencing ARDS, MOF, or sepsis is considered to have a favorable outcome. In some embodiments, a patient who recovers from trauma without experiencing ARDS, MODS, MOF, or sepsis is considered to have a favorable outcome. In some embodiments the methods of the invention result in a reduction in mortality, a reduction in incidence or severity of MOF (e.g., lower average number of failing organs), a reduction in incidence or severity of ARDS, and/or a reduced average length of time spent in an intensive care unit (ICU) among trauma survivors, in trauma patients treated according to an inventive method relative to trauma patients receiving conventional care.

The invention provides compositions and methods for modulating the complement system to reduce the likelihood of poor outcome following trauma. The present disclosure identifies, from among the many classes of available complement inhibitors, certain complement inhibitors that are most suitable for use in trauma patients. The invention further provides methods appropriate for the administration of such complement inhibitors so as to reduce the likelihood of poor outcome after trauma. Methods are provided that limit excessive complement activation in the immediate post-trauma period without detrimentally compromising the complement system's functional capacity. In some embodiments the methods limit excessive complement activation and help preserve the functional capacity of the complement system. In some embodiments, methods of the invention enhance the functional capacity of the complement system. An anti-depletion therapy administered in accordance with this invention inhibits complement activation and thereby inhibits consumption of complement components that would otherwise occur as a consequence of the injury. A pro-repletion therapy augments the functional capacity of the complement system, e.g., by administering one or more unactivated complement components to a subject. In some embodiments an anti-depletion therapy is administered within the first 24 hours after injury and/or a pro-repletion therapy is administered between 4 hours and 4 days after injury.

Complement System

To facilitate understanding the invention, and without intending to limit the invention in any way, this section provides an overview of complement and its pathways of activation. Further details are found in: Kuby Immunology, 2000; Paul, W. E., Fundamental Immunology, Lippincott Williams & Wilkins; 5^{th} ed., 2003; and Walport M J., Complement. First of two parts. N Engl J Med., 344(14):1058-66, 2001.

The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils.

The alternative pathway is initiated by, e.g., microbial surfaces and various complex polysaccharides. In this pathway, C3b, resulting from cleavage of C3, which occurs spontaneously at a low level, binds to targets, e.g., on cell surfaces and forms a complex with factor B, which is later cleaved by factor D, resulting in a C3 convertase. Cleavage of C3 and binding of another molecule of C3b to the C3 convertase gives rise to a C5 convertase. C3 and C5 convertases of this pathway are regulated by CR1, DAF (CD55), MCP(CD46), and CFH. The mode of action of these proteins involves either decay accelerating activity (i.e., ability to dissociate convertases), ability to serve as cofactors in the degradation of C3b or C4b by factor I, or both.

The C5 convertases produced in both pathways cleave C5 to produce C5a and CSb. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of adverse consequences other than cell death.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in proteolysis of C4 and C2, leading to a C3 convertase described above.

It has recently been proposed that some steps of complement activation such as cleavage of C3 and C5 can occur through the activity of certain proteolytic enzymes such as thrombin, kallikrein, and other serum proteases (the extrinsic protease pathway). See, e.g., Ricklin, D. and Lambris, J., Nat. Biotechnol., 25(11), 1265, 2007. As used herein, the term “protease of the extrinsic protease pathway” excludes those proteases that are complement components as defined above.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains. These domains, consisting of approximately 50-70 amino acids, typically about 60 amino acids, are characterized by a conserved motif that includes four disulfide-bonded cysteines (two disulfide bonds), proline, tryptophan, and many hydrophobic residues. The CCP family includes complement receptor type 1 (CR1; C3b:C4b receptor), complement receptor type 2 (CR2), membrane cofactor protein (MCP; CD46), decay-accelerating factor (DAF), complement factor H (1H), and C4b-binding protein (C4 bp). CD59 is a membrane-bound complement regulator unrelated structurally to the CCPs.

Complement Activation Inhibition as a Therapeutic Approach in Trauma

The present invention provides the recognition that inhibiting complement by administering a complement inhibitor having certain properties in the immediate post-trauma period (defined herein as the first 24 hours after occurrence of a traumatic injury) will reduce morbidity and mortality in trauma patients. The complement inhibitor should be capable both of inhibiting excessive complement activation and of limiting consumption of at least some complement components, e.g., C3, that would otherwise occur. Complement inhibitors particularly preferred for use in trauma have one, more than one, or all of the following properties: First, the complement inhibitor should inhibit complement activation. Such a complement inhibitor is referred to herein as a “complement activation inhibitor”. A complement activation inhibitor typically inhibits activity of a complement component that participates, e.g., as a substrate, protease, or protease subunit, in one or more of the biochemical cascades leading to formation of the C5bC6C7C8C9 complex via the classical, alternative, and/or MBL pathway. The term “complement activation inhibitor” excludes agents whose complement inhibiting activity is largely or essentially entirely limited to inhibiting certain cleavage fragments such as C2b, C3a, C4a, CSa, that do not participate in a biochemical cascade leading to formation of the C5bC6C7C8C9 or MAC complex. The term “complement activation inhibitor” also excludes agents whose complement inhibiting activity is largely or essentially entirely limited to inhibiting complement receptors such as C3aR, C4aR, C5aR, or CR1.

Second, the complement inhibitor should act at or above the level of C3 activation. A complement activation inhibitors acts “at or above the level of C3 activation” if it directly or indirectly inhibits activation of C3. The complement activation inhibitor may, for example, act directly or indirectly on C1, C2, C3, C4, fB, fD, or properdin. In the case of complement components that are inactive until they are cleaved or undergo hydrolysis, the complement activation inhibitor may bind to the intact, unactivated form and inhibit its cleavage or hydrolysis. The complement activation inhibitor may act directly or indirectly on an active fragment, complex, or subunit of any of the foregoing complement components that participates in one or more of the biochemical cascades leading to formation of a C3 convertase e.g., C1q, C1r, C1s, C2a, C3b, C4b, Bb. The complement activation inhibitor may directly or indirectly inhibit a C3 convertase, e.g., the classical or alternative pathway C3 convertase.

In some embodiments the complement activation inhibitor significantly inhibits activity of a protease of the extrinsic protease pathway. In other embodiments the complement activation inhibitor does not significantly inhibit a protease of the extrinsic protease pathway. In some embodiments the complement activation inhibitor does not comprise heparin, low molecular weight heparin, or a derivative thereof. In some embodiments the complement activation inhibitor does not comprise an agent known in the art to inhibit thrombin, kallikrein, or other plasma proteases of the extrinsic protease pathway. For example, in some embodiments the complement activation inhibitor is not a direct thrombin inhibitor such as hirudin, bivalirudin, ximelone, melagatran, desirudin or a structually related compound (Di Nisio, N. Engl. J. Med., 353L 10, 2005) or a kallikrein inhibitor such as those described in U.S. Pat. Pub. No. 20070049522). In some embodiments a complement activation inhibitor that acts directly on a complement component is administered in combination with an agent that inhibits the extrinsic protease pathway, e.g., one or more such agents is administered in combination with administering a preferred complement activation inhibitor.

Third, in some embodiments of the invention the complement inhibitor should not cause significant depletion or significant irreversible inhibition of an unactivated complement component at a time point 48 hours, or in some embodiments 24 hours or in some embodiments 12 hours, after administration of the complement inhibitor (and thereafter). In some embodiments, the complement inhibitor should not cause extensive depletion or extensive irreversible inhibition of an unactivated complement component at a time point 48 hours, or in some embodiments 24 hours or in some embodiments 12 hours, after administration of the complement inhibitor. In some embodiments, the complement inhibitor should cause no more than minimal depletion or irreversible inhibition of an unactivated complement component at a time point 48 hours, or in some embodiments 24 hours or in some embodiments 12 hours, after administration of the complement inhibitor. As used herein, an agent is said to cause depletion or irreversible inhibition of an unactivated complement component if one or more of the following occurs: (i) the agent covalently binds to the complement component and the covalently bound form is inactive and incapable of being activated; (ii) the agent noncovalently binds to the complement component and causes a change in its structure (e.g., tertiary structure) that does not activate and renders the complement component incapable of being activated, wherein the change in structure does not reverse upon dissociation of the agent; (iii) the agent causes a non-activating chemical change to the component (e.g., non-activating cleavage) and thereby renders it incapable of being activated; (iv) the agent binds to the complement component and the resulting complex is cleared or eliminated by the body before dissociation occurs. The foregoing list is non-limiting. In some embodiments of the invention a complement component is considered “incapable of being activated” if its likelihood of becoming active is reduced by at least 95%, 98%, 99%, or more, e.g., by 100% (i.e., to undetectable levels), under conditions in which activation would otherwise be expected to measurably occur. “Significant depletion or significant irreversible inhibition” refers to depletion or irreversible inhibition by more than 25% (e.g., the agent has acted in at least one of the foregoing ways on at least 25% of the target molecules that would otherwise be present and activatable). “Extensive depletion or extensive irreversible inhibition” refers to depletion or irreversible inhibition by more than 50% (e.g., the agent has acted in one or more of the foregoing ways on at least 50% of the target molecules that would otherwise be present and activatable). “No more than minimal depletion or irreversible inhibition” refers to depletion or irreversible inhibition by no more than 10% (e.g., the agent has acted in one or more of the foregoing ways on no more than 10% of the target molecules that would otherwise be present and activatable).

The invention provides a method of treating an individual who has suffered a traumatic injury comprising administering a complement activation inhibitor to the individual in an amount sufficient such that 24 hours after the injury the concentration of intact (uncleaved) C3 in the individual's blood is greater than would have been the case had the complement activation inhibitor not been administered. The invention provides a method of treating an individual who has suffered a traumatic injury comprising administering a complement activation inhibitor to the individual in an amount sufficient such that 24 hours after the injury the concentration of intact (uncleaved) C3 in the individual's blood is greater than the average concentration of intact C3 present 24 hours after injury in the blood of individuals having an injury of similar severity to whom a complement activation inhibitor has not been administered within the first 24 hours after occurrence of the injury. The invention provides a method of treating an individual who has suffered a traumatic injury comprising administering a complement activation inhibitor to the individual in an amount sufficient such that 24 hours after the injury the concentration of intact (uncleaved) C5 in the individual's blood is greater than would have been the case had the complement activation inhibitor not been administered. The invention provides a method of treating an individual who has suffered a traumatic injury comprising administering a complement activation inhibitor to the individual in an amount sufficient such that 24 hours after the injury the concentration of intact (uncleaved) C5 in the individual's blood is greater than the average concentration of intact C5 present 24 hours after injury in the blood of individuals having an injury of similar severity to whom a complement activation inhibitor has not been administered within the first 24 hours after occurrence of the injury.

In certain embodiments of the invention a complement activation inhibitor inhibits activation of a complement component, and 48 hours after administration of the complement activation inhibitor to a normal subject, or in some embodiments 24 hours after administration, or in some embodiments 12 hours after administration, at least 50%, or in some embodiments at least 75%, or in some embodiments at least 90%, of the molecules of the complement component that would have been present in the subject's blood in activatable form in the absence of the complement activation inhibitor, are present and are potentially activatable. To assess whether this is the case, a blood sample can be obtained from a subject prior to and after administration of a complement inhibitor (e.g., 12, 24, or 48 hours after administration). Optionally the samples are supplemented with one or complement components other than the one to be assessed so that the complement component to be assessed is present in limiting quantities. An assay that measures functional complement activity is performed on the samples. If the result shows at least 50%, 75%, or 90% activity in the sample obtained after administration relative to that observed in the sample obtained prior to administration, one can conclude that at least 50%, 75%, or 90%, respectively, of the molecules of the complement component that would have been present in the subject's blood in activatable form in the absence of the complement inhibitor are present in activatable form.

In certain embodiments of the invention the foregoing properties are assessed based on the assumption that the complement activation inhibitor is administered in an amount effective to transiently inhibit systemic complement activation or activity in a normal subject by at least 75% and/or in an amount sufficient such that activation of at least 75% of the molecules of the complement component in the systemic circulation in a normal subject are transiently inhibited. In certain embodiments of the invention the foregoing properties are assessed based on the assumption that the complement activation inhibitor is administered in an amount effective to transiently inhibit systemic complement activation or activity in a normal subject by at least 90% and/or in an amount sufficient such that activation of at least 90% of the molecules of the complement component in the systemic circulation in a normal subject are transiently inhibited. In some embodiments of the invention transient inhibition persists for at least 1, 2, 4, 6, 8, 12, or 24 hours, e.g., between 1 and 24 hours or any intervening range.

In certain embodiments of the invention a complement activation inhibitor has the following property with regard to a complement component whose activation it inhibits: after (i) contacting the complement activation inhibitor with the complement component in vitro under conditions suitable for the complement activation inhibitor to physically interact with and inhibit the complement component (which in some embodiments comprise using at least an equal amount of complement inhibitor, e.g., at least 2, 5, or 10 times as much complement inhibitor as complement component on a molar basis); (ii) isolating complexes comprising the complement component and the inhibitor, e.g., at a time between 5 minutes and 6 hours after initial contacting; and (iii) maintaining the complement component complexes isolated in step (ii) for 48 hours, or in some embodiments 24 hours, in a liquid medium under conditions consistent with maintaining activatability of the complement component (e.g., in a liquid medium optionally having about pH 6-8, about 37 degrees C., about physiological salt concentration, optionally containing osmotic or protein stabilizing agents that do not interfere with complement activation, and not containing complement activation inhibitor other than as present in the complex), at least 50%, at least 75%, or at least 90% of the molecules of the complement component that would have been activatable had the same amount of complement component as present in the complex been maintained and isolated under the same conditions of (i)-(iii) but without the presence of the complement activation inhibitor, remain activatable notwithstanding having been contacted with the complement activation inhibitor. It will be appreciated that the volume of liquid medium used in step (iii) should be sufficiently large that the likelihood of a complement component molecule encountering a second molecule of the complement activation inhibitor after having dissociated from the complex is low (e.g., less than 10% over the duration of the experiment). Alternately, the vessel in which step (iii) is performed could be such that the complement activation inhibitor could be removed from the vessel (e.g., by diffusion across a membrane such as a dialysis membrane having pores that permit diffusion of the inhibitor but not diffusion of the complement component). Thus complement inhibitors may be tested and/or compared in vitro to determine which among the various agents availablehave one or more of the afore-mentioned properties. Such testing methods are aspects of the invention.

In some embodiments of the invention, a complex comprising the complement activation inhibitor and the complement component dissociates with a half-life of between 1 and 24 hours in vitro under conditions consistent with maintaining activatability of the complement component, and at least 50%, at least 75%, or at least 90% of the released complement component molecules remain activatable and/or at least 50%, at least 75%, or at least 90% of the overall activatability of the dissociated complement component molecules collectively after such period is retained.

In some embodiments of the invention the complement inhibitor acts by sterically hindering the access of a substate complement component by a convertase that would otherwise cleave and activate it.

In certain embodiments of the invention the complement activation inhibitor is administered within 1, 2, 3, 4, 6, 8, 12, or 24 hours following injury. In some embodiments the complement inhibitor is administered essentially immediately following injury (i.e., within less than 5 minutes following injury). In some embodiments the complement inhibitor is administered at least 5 minutes following injury but within the first 30 minutes following injury, or in some embodiments within the first 1 hour following injury, or in some embodiments within 2, 3, 4, 6, 8, 12, or 24 hours after injury. In some embodiments the complement activation inhibitor is administered multiple times within any of the afore-mentioned time intervals. In certain embodiments the complement activation inhibitor is not administered in active form (e.g., in a form capable of inhibiting activation of complement upon administration) more than 12 hours or, in some embodiments, more than 24 hours, after injury. For example, the complement activation inhibitor is administered in active form one or more times within 12 hours or less or within 24 hours or less after injury and is not administered thereafter in active form in amounts sufficient to significantly inhibit complement activation for at least 10 days (e.g., not within 2 weeks, 4 weeks, or 6 weeks thereafter).

In some embodiments of the invention, the complement activation inhibitor is administered in a complex with a complement component at one or more time points at least 4 hours after the injury, e.g., at least 12 hours, at least 24 hours, at least 48 hours, at least 72 hours, between 48 hours and 2 weeks, etc.

Certain preferred complement activation inhibitors administered according to certain embodiments of the present invention, by intervening in the complement activation cascade at or above the level of C3, both inhibit complement activation and reduce consumption of complement components that would otherwise occur as a consequence of complement activation in a trauma patient who has suffered a severe injury. The inventive approach not only inhibits complement activation in the trauma patient during at least part of the immediate post-trauma period, but also at least in part preserves and in some embodiments enhances the patient's capacity to activate complement at later time points, e.g., at least 24 hours after trauma.

In some embodiments a sufficient dose (or multiple doses) of complement activation inhibitor is administered to reduce complement activation (e.g., as measured by the level of C3 split products in the blood or any other method useful for measuring complement activation) and/or to reduce complement component depletion in the trauma patient by at least 50%, at least 75%, or at least 90% relative to the level that would occur in the absence of the complement activation inhibitor, when assessed at a time point sufficiently long after administration of the complement activation inhibitor.

In certain embodiments of the invention the complement activation inhibitor is selected such that, if administered to a normal subject in an amount sufficient to reduce systemic complement activation capacity by at least 75% for at least 5 minutes, the subject's complement activation capacity would have returned to at least 75% of pre-administration level 12 hours, or in some embodiments 24 hours, or in some embodiments 48 hours, after administration. In certain embodiments of the invention the complement activation inhibitor is selected such that, if administered to a normal subject in an amount sufficient to reduce systemic complement activation capacity by at least 75% for at least 5 minutes, the subject's complement activation capacity would have returned to at least 90% of pre-administration level 12 hours, or in some embodiments 24 hours, or in some embodiments 48 hours, after administration. In certain embodiments of the invention the complement activation inhibitor and dose are selected such that, if administered to a normal subject in an amount sufficient to reduce systemic complement activation capacity by at least 75% for at least 5 minutes, the subject's systemic complement activation capacity would have returned to at least 75% of pre-administration level 12 hours, or in some embodiments 24 hours, or in some embodiments 48 hours, after administration. “Complement activation capacity”, also referred to as “potential complement activity” refers to the level of complement activation that would occur if the subject or a blood or plasma sample obtained from the subject was exposed to a stimulus that causes maximum complement activation. It may be measured using, e.g., a suitable functional assay such as an assay based on hemolysis, complement deposition, etc. Pathway-specific complement activation capacity may be assessed using, e.g., appropriate stimuli and conditions to activate one or more than one of the pathways.

In some embodiments, the complement activation inhibitor is administered in an amount such that, by 72 hours after the injury, or in some embodiments by 48 hours after the injury, or in some embodiments by 12 hours after the injury, or in some embodiments by 4 hours after the injury, the individual's potential classical pathway activity, alternative pathway activity, or both, is/are significantly preserved at least in part as a result of administering the complement activation inhibitor. “Significantly preserved” in this context means, in various embodiments of the invention, that the capacity of the subject to activate complement by the classical or alternative pathway, respectively, is at least 25%, at least 50%, at least 75%, at least 90%, or any intervening range between 20% and 100%, of an average, normal value (e.g., what it would have been if the injury had not occurred).

In some embodiments of the invention the complement activation inhibitor has a half-life of 24 hours or less in humans when administered intravenously. In some embodiments of the invention the complement inhibitor has a half-life of 12 hours or less in humans when administered intravenously. In some embodiments of the invention the complement inhibitor has a half-life of 6 hours or less in humans when administered intravenously. In some embodiments of the invention the complement inhibitor has a half-life of 2 hours or less in humans when administered intravenously. In some embodiments of the invention the complement inhibitor has a half-life of 1 hour or less in humans when administered intravenously. The half-life will typically be at least 5 minutes.

In certain embodiments of the invention the complement activation inhibitor inhibits the alternate pathway. In certain embodiments of the invention the complement activation inhibitor inhibits the classical pathway. In some embodiments the complement inhibitor inhibits the classical and alternative pathways. In some embodiments of the invention the complement activation inhibitor inhibits all three major complement activation pathways.

The invention further provides a method of treating a trauma patient comprising: administering first and second complement activation inhibitors to the patient, wherein the first complement activation inhibitor inhibits C3 activation and the second complement activation inhibitor inhibits C5 cleavage. In certain embodiments of the invention the complement activation inhibitors are administered as a single composition.

In some embodiments of the invention the complement activation inhibitor is first administered “in the field”, i.e., outside the setting of a health care facility, e.g., at or near the location where the event responsible for the injury occurred or during transport of the patient to a health care facility. In some embodiments the complement activation inhibitor is first administered in an emergency room. In some embodiments of the invention a single dose of the complement activation inhibitor is administered. In some embodiments of the invention complement activation inhibitor therapy is halted no more than 12 hours after the injury. In some embodiments of the invention complement activation inhibitor therapy is halted no more than 24 hours after the injury.

Typically, the complement activation inhibitor is administered intravenously although other routes of administration may be used in certain embodiments of the invention. In some embodiments of the invention the complement activation inhibitor is administered as an intravenous (IV) bolus. In some embodiments the complement activation inhibitor is administered by IV infusion. In some embodiments of the invention the complement activation inhibitor is administered as an IV bolus followed by an IV infusion. In some embodiments, the method comprises administering the complement activation inhibitor as an IV drip. An early step in treating a trauma victim is often placing an IV line and providing IV fluids. In some embodiments the complement activation inhibitor is added to such IV fluid.

The invention provides fixed dose formulations containing sufficient complement activation inhibitor to significantly inhibit complement activation in a child or adult human following a single administration. In some embodiments of the invention the formulation reduces systemic complement activity by between 50% and 99%, e.g., by at least 50%, 75%, or 90%, relative to levels present prior to administration. In some embodiments of the invention the formulation reduces systemic complement activity by between 50% and 99%, e.g., by at least 50%, 75%, or 90% relative to normal, average levels. In some embodiments of the invention the complement activation inhibitor binds to an unactivated complement component, and the formulation contains sufficient complement activation inhibitor to bind to at least 75%, at least 90%, at least 150%, at least 250%, or at least 500% of the molecules of said complement component found in an average adult human.

Patient Selection and Characteristics

In some embodiments of the invention, an individual who has suffered a traumatic injury is assessed prior to administration of a complement activation inhibitor, e.g., the severity of the injury is assessed using a trauma score (i.e., a scoring system that provides an indication of the extent of injury of a trauma victim). A variety of established injury scoring systems, or scoring systems developed for purposes of the present invention, may be used. In some embodiments of the invention the scording system provides a quantitative indicator of injury severity. The Revised Trauma Score (RTS) is a physiological scoring system, with high inter-rater reliability and demonstrated accuracy in predicting death. It is scored from the first set of data obtained on the patient, and consists of Glasgow Coma Scale, systolic blood pressure and respiratory rate (Champion H R et al, “A Revision of the Trauma Score”, J Trauma 29:623-629, 1989; Champion H R et al, “Trauma Score”, Crit Care Med 9:672-676, 1981). Values for the RTS are computed using the formula RTS=0.9368 GCS+0.7326 SBP+0.2908 RR and are typically in the range 0 to 7.8408. The Injury Severity Score (ISS) is an anatomical scoring system that provides an overall score for patients with multiple injuries Baker S P et al, “The Injury Severity Score: a method for describing patients with multiple injuries and evaluating emergency care”, J Trauma 14:187-196; 1974. Each injury is assigned an Abbreviated Injury Scale (AIS) score and is allocated to one of six body regions (Head, Face, Chest, Abdomen, Extremities (including Pelvis), External). Only the highest AIS score in each body region is used. The 3 most severely injured body regions have their score squared and added together to produce the ISS score. The ISS score ranges from 0 to 75. If an injury is assigned an AIS of 6 (unsurvivable injury), the ISS score is automatically assigned to 75. The ISS score is the most widely used anatomical scoring system and correlates linearly with mortality, morbidity, hospital stay and other measures of severity. In some embodiments of the invention the complement activation inhibitor is administered to an individual having an ISS of at least 30, e.g., between 30 and 75 or any intervening range. In some embodiments of the invention the complement activation inhibitor is administered to an individual having an ISS of at least 15. In some embodiments the complement activation inhibitor is administered to an individual having an ISS of at least 9. In some embodiments the individual has an RTS of at least 4.

Injuries may be considered “of similar severity” based on a trauma score (see discussion below). For example, individuals having a trauma score falling within a 10% subrange, or in some embodiments a 20% subrange, or in some embodiments a 30% subrange of a trauma score may be considered to have injuries of similar severity. In some embodiments individuals having a trauma score above a particular threshold trauma score are considered to have injuries of similar severity.

The invention provides a method of treating an individual who has suffered a traumatic injury comprising steps of: (a) assessing the severity of the traumatic injury within 1, 2, 4, 6, or 12 hours following occurrence of the injury; and (b) administering a complement inhibitor to the individual if the severity of the injury is above a predetermined level. In certain embodiments step (a) comprises determining a trauma score. In some embodiments the severity is assessed before the individual arrives at a health care facility such as a hospital. The assessment can take place at the location where the event causing the injury occurred or in a transport vehicle such as an ambulance or helicopter or in the emergency department of a health care facility, etc.

In some embodiments of the invention a patient is determined to be at increased risk of poor outcome following trauma as a result of having particular genotype with respect to one or more genes encoding a complement-related protein, e.g., gene encoding CFH, fB, C2, C3, etc. The invention encompasses assessing the genotype of the patient with regard to one or more complement-related genes and deciding at least in part based on the assessment whether to administer a complement activation inhibitor and/or selecting a particular agent and/or dose. See, e.g., PCT/US2008/070650, for discussion of various polymorphisms of interest. “Assessing” encompasses receiving information regarding the genotype wherein the genotype may have been determined prior to or after the injury. Optionally a genotyping assay is performed in the field or upon arrival at a health care facility.

In some embodiments of the invention the trauma patient is at least 18 years of age. In some embodiments of the invention the trauma patient is less than 18 years of age. In some embodiments of the invention the trauma patient is male. In some embodiments of the invention the trauma patient is female. In some embodiments the patient has suffered trauma to the head or spinal column and has or is at increased risk of traumatic brain injury or spinal cord injury. In some embodiments the subject has suffered thermal injury. In some embodiments the subject has suffered inhalation injury. In some embodiments the subject has suffered significant blood loss and is at risk of or suffers from ischemia-reperfusion injury. In some embodiments the subject has suffered injury of sufficient severity that admission to an ICU is indicated.

In some embodiments of the invention the individual does not have and/or has not been previously diagnosed with a condition, other than the injury, for which complement activation inhibition would be indicated. In some embodiments the individual does not have and/or has not previously been diagnosed with a condition, other than the injury, for which systemic complement activation inhibition would be indicated. Usually the individual does not have a condition, other than the injury, for which complement repletion therapy would be indicated. In some embodiments of the invention an individual has or has previously been diagnosed with a complement-mediated disorder or complement deficiency, but the inventive trauma therapy utilizes a different composition or dosing regimen than would be indicated for treating such disorder or deficiency.

Complement Activation Inhibitors

The following sections describe complement activation inhibitors and compositions containing them provided by the present invention and/or useful in the practice of the inventive methods. Complement inhibitors for use in the present inventive methods may be selected from among the various classes of complement inhibitors described in this section, based on properties such as those described above (e.g., half-life, performance in the in vitro tests described above, etc.), and the invention provides embodiments specifically directed to those complement inhibitors having properties described above. Furthermore, the invention provides embodiments in which, for any particular complement inhibitor that acts at or above the level of C3 activation the complement inhibitor is administered within any of the time intervals mentioned above. The invention provides embodiments in which, for any particular complement inhibitor that acts at or above the level of C3 activation, the complement inhibitor is administered in combination with an agent that inhibits cleavage of C5.

Compstatin Analogs

Compstatin is a cyclic peptide that binds to complement component C3 and inhibits complement activation. Compstatin inhibits cleavage of C3 to C3a and C3b by convertase. Since C3 is a central component of all three pathways of complement activation, compstatin and analogs thereof are able to inhibit all three pathways. U.S. Pat. No. 6,319,897 describes a peptide having the sequence Ile-[Cys-Val-Val-Gln-Asp-Trp-Gly-His-His-Arg-Cys]-Thr (SEQ ID NO: 41 herein, SEQ ID NO: 2 in the afore-mentioned patent), with the disulfide bond between the two cysteines denoted by brackets. The name “compstatin” was not used in U.S. Pat. No. 6,319,897 but was subsequently adopted in the scientific and patent literature (see, e.g., Morikis, et al., Protein Sci., 7(3):619-27, 1998) to refer to a peptide having the same sequence as SEQ ID NO: 2 disclosed in U.S. Pat. No. 6,319,897, but amidated at the C terminus as shown in Table 1 (SEQ ID NO: 8). The term “compstatin” is used herein consistently with such usage (i.e., to refer to SEQ ID NO: 8).

Compstatin analogs that have higher complement inhibiting activity than compstatin have been developed. See, e.g., WO2004/026328 (PCT/US2003/029653), Morikis, D., et al., Biochem Soc Trans. 32(Pt 1):28-32, 2004, Mallik, B., et al., J. Med. Chem., 274-286, 2005; Katragadda, M., et al. J. Med. Chem., 49: 4616-4622, 2006; WO2007062249 (PCT/US2006/045539); WO2007044668 (PCT/US2006/039397), and discussion below. As used herein, the term “compstatin analog” includes compstatin and any complement inhibiting analog thereof designed or based on compstatin whose complement inhibiting activity is at least 50% as great as that of compstatin as measured, e.g., using any complement activation assay accepted in the art or substantially similar or equivalent assays. The assay may, for example, measure alternative pathway-mediated erythrocyte lysis. Antibody-based assays such as ELISA assays are also of use. Such assays may detect complement deposition and/or complement activation products. WO2004/026328, Morikis, supra, Mallik, supra, and Katragadda 2006, supra, among other references, describe suitable methods for assessing the ability of a compound to inhibit complement activation.

The activity of a compstatin analog may be expressed in terms of its IC₅₀ (the concentration of the compound that inhibits complement activation by 50%), e.g., at a particular plasma concentration, with a lower IC₅₀ indicating a higher activity as recognized in the art. The activity of a compstatin analog for use in certain embodiments of the present invention is at least as great as that of compstatin. Certain modifications are known to reduce or eliminate complement inhibiting activity and may be explicitly excluded from any embodiment of the invention. It will be appreciated that the precise IC₅₀ value measured for a given compstatin analog will vary with experimental conditions. Comparative values, e.g., obtained from experiments in which IC₅₀ is determined for multiple different compounds under substantially identical conditions, are of use. In one embodiment, the IC₅₀ of the compstatin analog is no more than the IC₅₀ of compstatin. In certain embodiments the IC₅₀ of the compstatin analog is between about 0.2 μM and about 0.5 μM. In certain embodiments the ICₛₒ of the compstatin analog is between about 0.1 μM and about 0.2 μM. In certain embodiments the IC₅₀ of the compstatin analog is between about 0.05 μM and about 0.1 μM. In certain embodiments the IC₅₀ of the compstatin analog is between about 0.001 μM and about 0.05 μM.

In certain embodiments of the invention the compstatin analog is not compstatin. In certain embodiments of the invention the activity of the compstatin analog is greater than that of compstatin, e.g., between 2 and 99 times that of compstatin (e.g., the analog has an IC₅₀ that is less than the IC₅₀ of compstatin by a factor of between 2 and 99). For example, the activity may be between 10 and 50 times as great as that of compstatin, or between 50 and 99 times as great as that of compstatin. In certain embodiments of the invention the activity of the compstatin analog is between 99 and 264 times that of compstatin. For example, the activity may be 100, 110, 120, 130, 140, 150, 160, 170, 180, 190, 200, 210, 220, 230, 240, 250, 260, or 264 times as great as that of compstatin. In certain embodiments of the invention the compstatin analog is a potent compstatin analog such as an analog having an activity at least 100-fold greater than that of compstatin. In certain embodiments of the invention the activity of the compstatin analog is at least 150, at least 200, or at least 250 times as great as that of compstatin. Table 1 presents sequences of a number of such analogs. In certain embodiments the invention contemplates use of analogs whose activity is between 264 and 300, 300 and 350, 350 and 400, or 400 and 500 times as great as that of compstatin. The invention further contemplates use of compstatin analogs having activities between 500 and 1000 times that of compstatin.

The K_{d} of compstatin binding to C3 has been measured as 1.3 μM using isothermal titration calorimetry (Katragadda, et al., J. Biol. Chem., 279(53), 54987-54995, 2004) wherein, the term “compstatin” was used to refer to the acetylated version of SEQ ID NO: 8, i.e., SEQ ID NO: 9. Binding affinity of a variety of compstatin analogs for C3 has been correlated with their activity, with a lower K_{d} indicating a higher binding affinity, as recognized in the art. A linear correlation between binding affinity and activity was shown for certain analogs tested (Katragadda, 2004, supra; Katragadda 2006, supra). In certain embodiments of the invention the compstatin analog binds to C3 with a K_{d} of between 0.1 μM and 1.0 μM, between 0.05 μmM and 0.1 μM, between 0.025 μM and 0.05 μM, between 0.015 μM and 0.025 μM, between 0.01 μM and 0.015 μM, or between 0.001 μM and 0.01 μM.

Compounds “designed or identified based on compstatin” include, but are not limited to, compounds that comprise an amino acid chain whose sequence is obtained by (i) modifying the sequence of compstatin (e.g., replacing one or more amino acids of the sequence of compstatin with a different amino acid or amino acid analog, inserting one or more amino acids or amino acid analogs into the sequence of compstatin, or deleting one or more amino acids from the sequence of compstatin); (ii) selection from a phage display peptide library in which one or more amino acids of compstatin is randomized, and optionally further modified according to method (i); or (iii) identified by screening for compounds that compete with compstatin or any analog thereof obtained by methods (i) or (ii) for binding to C3 or a fragment thereof. Many useful compstatin analogs comprise a hydrophobic cluster, a β-turn, and a disulfide bridge.

In certain embodiments of the invention the sequence of the compstatin analog comprises or consists essentially of a sequence that is obtained by making 1, 2, 3, or 4 substitutions in the sequence of compstatin, i.e., 1, 2, 3, or 4 amino acids in the sequence of compstatin is replaced by a different standard amino acid or by a non-standard amino acid. In certain embodiments of the invention the amino acid at position 4 is altered. In certain embodiments of the invention the amino acid at position 9 is altered. In certain embodiments of the invention the amino acids at positions 4 and 9 are altered. In certain embodiments of the invention only the amino acids at positions 4 and 9 are altered. In certain embodiments of the invention the amino acid at position 4 or 9 is altered, or in certain embodiments both amino acids 4 and 9 are altered, and in addition up to 2 amino acids located at positions selected from 1, 7, 10, 11, and 13 are altered. In certain embodiments of the invention the amino acids at positions 4, 7, and 9 are altered. In certain embodiments of the invention amino acids at position 2, 12, or both are altered, provided that the alteration preserves the ability of the compound to be cyclized. Such alteration(s) at positions 2 and/or 12 may be in addition to the alteration(s) at position 1, 4, 7, 9, 10, 11, and/or 13. Optionally the sequence of any of the compstatin analogs whose sequence is obtained by replacing one or more amino acids of compstatin sequence further includes up to 1, 2, or 3 additional amino acids at the C-terminus In one embodiment, the additional amino acid is Gly. Optionally the sequence of any of the compstatin analogs whose sequence is obtained by replacing one or more amino acids of compstatin sequence further includes up to 5, or up to 10 additional amino acids at the C-terminus. It should be understood that compstatin analogs may have any one or more of the characteristics or features of the various embodiments described herein, and characteristics or features of any embodiment may additionally characterize any other embodiment described herein, unless otherwise stated or evident from the context.

In certain embodiments of the invention the sequence of the compstatin analog comprises or consists essentially of a sequence that is obtained by making 1, 2, 3, or 4 substitutions in the sequence of compstatin, i.e., 1, 2, 3, or 4 amino acids in the sequence of compstatin is replaced by a different standard amino acid or by a non-standard amino acid. In certain embodiments of the invention the amino acid at position 4 is altered. In certain embodiments of the invention the amino acid at position 9 is altered. In certain embodiments of the invention the amino acids at positions 4 and 9 are altered. In certain embodiments of the invention only the amino acids at positions 4 and 9 are altered. In certain embodiments of the invention the amino acid at position 4 or 9 is altered, or in certain embodiments both amino acids 4 and 9 are altered, and in addition up to 2 amino acids located at positions selected from 1, 7, 10, 11, and 13 are altered. In certain embodiments of the invention the amino acids at positions 4, 7, and 9 are altered. In certain embodiments of the invention amino acids at position 2, 12, or both are altered, provided that the alteration preserves the ability of the compound to be cyclized. Such alteration(s) at positions 2 and/or 12 may be in addition to the alteration(s) at position 1, 4, 7, 9, 10, 11, and/or 13. Optionally the sequence of any of the compstatin analogs whose sequence is obtained by replacing one or more amino acids of compstatin sequence further includes up to 1, 2, or 3 additional amino acids at the C-terminus In one embodiment, the additional amino acid is Gly. Optionally the sequence of any of the compstatin analogs whose sequence is obtained by replacing one or more amino acids of compstatin sequence further includes up to 5, or up to 10 additional amino acids at the C-terminus. It should be understood that compstatin analogs may have any one or more of the characteristics or features of the various embodiments described herein, and characteristics or features of any embodiment may additionally characterize any other embodiment described herein, unless otherwise stated or evident from the context. In certain embodiments of the invention the sequence of the compstatin analog comprises or consists essentially of a sequence identical to that of compstatin except at positions corresponding to positions 4 and 9 in the sequence of compstatin.

Compstatin and certain compstatin analogs having somewhat greater activity than compstatin contain only standard amino acids (“standard amino acids” are glycine, leucine, isoleucine, valine, alanine, phenylalanine, tyrosine, tryptophan, aspartic acid, asparagine, glutamic acid, glutamine, cysteine, methionine, arginine, lysine, proline, serine, threonine and histidine). Certain compstatin analogs having improved activity incorporate one or more non-standard amino acids. Useful non-standard amino acids include singly and multiply halogenated (e.g., fluorinated) amino acids, D-amino acids, homo-amino acids, N-alkyl amino acids, dehydroamino acids, aromatic amino acids (other than phenylalanine, tyrosine and tryptophan), ortho-, meta- or para-aminobenzoic acid, phospho-amino acids, methoxylated amino acids, and α,α-disubstituted amino acids. In certain embodiments of the invention, a compstatin analog is designed by replacing one or more L-amino acids in a compstatin analog described elsewhere herein with the corresponding D-amino acid. Such compounds and methods of use thereof are an aspect of the invention. Exemplary non-standard amino acids of use include 2-naphthylalanine (2-NaI), 1-naphthylalanine (1-NaI), 2-indanylglycine carboxylic acid (2Ig1), dihydrotrpytophan (Dht), 4-benzoyl-L-phenylalanine (Bpa), 2-α-aminobutyric acid (2-Abu), 3-α-aminobutyric acid (3-Abu), 4-α-aminobutyric acid (4-Abu), cyclohexylalanine (Cha), homocyclohexylalanine (hCha), 4-fluoro-L-tryptophan (4fW), 5-fluoro-L-tryptophan (5fW), 6-fluoro-L-tryptophan (6fW), 4-hydroxy-L-tryptophan (4OH—W), 5-hydroxy-L-tryptophan (5OH—W), 6-hydroxy-L-tryptophan (6OH—W), 1-methyl-L-tryptophan (1MeW), 4-methyl-L-tryptophan (4MeW), 5-methyl-L-tryptophan (SMeW), 7-aza-L-tryptophan (7aW), α-methyl-L-tryptophan (αMeW), β-methyl-L-tryptophan (βMeW), N-methyl-L-tryptophan (NMeW), ornithine (orn), citrulline, norleucine, γ-glutamic acid, etc.

In certain embodiments of the invention the compstatin analog comprises one or more Trp analogs (e.g., at position 4 and/or 7 relative to the sequence of compstatin). Exemplary Trp analogs are mentioned above. See also Beene, et. al. Biochemistry 41: 10262-10269, 2002 (describing, inter alia, singly- and multiply-halogenated Trp analogs); Babitzke & Yanofsky, J. Biol. Chem. 270: 12452-12456, 1995 (describing, inter alia, methylated and halogenated Trp and other Trp and indole analogs); and U.S. Pat. Nos. 6,214,790, 6,169,057, 5,776,970, 4,870,097, 4,576,750 and 4,299,838. Other Trp analogs include variants that are substituted (e.g., by a methyl group) at the α or β carbon and, optionally, also at one or more positions of the indole ring Amino acids comprising two or more aromatic rings, including substituted, unsubstituted, or alternatively substituted variants thereof, are of interest as Trp analogs. In certain embodiments of the invention the Trp analog, e.g., at position 4, is 5-methoxy, 5-methyl-, 1-methyl-, or 1-formyl-tryptophan. In certain embodiments of the invention a Trp analog (e.g., at position 4) comprising a 1-alkyl substituent, e.g., a lower alkyl (e.g., C₁-C₅) substituent is used. In certain embodiments, N(α) methyl tryptophan or 5-methyltryptophan is used. In some embodiments, an analog comprising a 1-alkanyol substituent, e.g., a lower alkanoyl (e.g., C₁-C₅) is used. Examples include 1-acetyl-L-tryptophan and L-β-tryptophan.

In certain embodiments the Trp analog has increased hydrophobic character relative to Trp. For example, the indole ring may be substituted by one or more alkyl (e.g., methyl) groups. In certain embodiments the Trp analog participates in a hydrophobic interaction with C3. Such a Trp analog may be located, e.g., at position 4 relative to the sequence of compstatin. In certain embodiments the Trp analog comprises a substituted or unsubstituted bicyclic aromatic ring component or two or more substituted or unsubstituted monocyclic aromatic ring components. In certain embodiments the Trp analog has increased propensity to form hydrogen bonds with C3 relative to Trp but does not have increased hydrophobic character relative to Trp. The Trp analog may have increased polarity relative to Trp and/or an increased ability to participate in an electrostatic interaction with a hydrogen bond donor on C3. Certain exemplary Trp analogs with an increased hydrogen bond forming character comprise an electronegative substituent on the indole ring. Such a Trp analog may be located, e.g., at position 7 relative to the sequence of compstatin.

In certain embodiments of the invention the compstatin analog comprises one or more Ala analogs (e.g., at position 9 relative to the sequence of compstatin), e.g., Ala analogs that are identical to Ala except that they include one or more CH₂ groups in the side chain. In certain embodiments the Ala analog is an unbranched single methyl amino acid such as 2-Abu. In certain embodiments of the invention the compstatin analog comprises one or more Trp analogs (e.g., at position 4 and/or 7 relative to the sequence of compstatin) and an Ala analog (e.g., at position 9 relative to the sequence of compstatin).

In certain embodiments of the invention the compstatin analog is a compound that comprises a peptide that has a sequence of (X′aa)ₙ-Gln-Asp-Xaa-Gly-(X″aa)ₘ, (SEQ ID NO: 2) wherein each X′aa and each X″aa is an independently selected amino acid or amino acid analog, wherein Xaa is Trp or an analog of Trp, and wherein n>1 and m>1 and n+m is between 5 and 21. The peptide has a core sequence of Gln-Asp-Xaa-Gly, where Xaa is Trp or an analog of Trp, e.g., an analog of Trp having increased propensity to form hydrogen bonds with an H-bond donor relative to Trp but, in certain embodiments, not having increased hydrophobic character relative to Trp. For example, the analog may be one in which the indole ring of Trp is substituted with an electronegative moiety, e.g., a halogen such as fluorine. In one embodiment Xaa is 5-fluorotryptophan. Absent evidence to the contrary, one of skill in the art would recognize that any non-naturally occurring peptide whose sequence comprises this core sequence and that inhibits complement activation and/or binds to C3 will have been designed based on the sequence of compstatin. In an alternative embodiment Xaa is an amino acid or amino acid analog other than a Trp analog that allows the Gln-Asp-Xaa-Gly peptide to form a β-turn.

In certain embodiments of the invention the peptide has a core sequence of X′aa-Gln-Asp-Xaa-Gly (SEQ ID NO: 3), where X′aa and Xaa are selected from Trp and analogs of Trp. In certain embodiments of the invention the peptide has a core sequence of X′aa-Gln-Asp-Xaa-Gly (SEQ ID NO: 3), where X′aa and Xaa are selected from Trp, analogs of Trp, and other amino acids or amino acid analogs comprising at least one aromatic ring. In certain embodiments of the invention the core sequence forms a β-turn in the context of the peptide. The β-turn may be flexible, allowing the peptide to assume two or more conformations as assessed for example, using nuclear magnetic resonance (NMR). In certain embodiments X′aa is an analog of Trp that comprises a substituted or unsubstituted bicyclic aromatic ring component or two or more substituted or unsubstituted monocyclic aromatic ring components. In certain embodiments of the invention X′aa is selected from the group consisting of 2-napthylalanine, 1-napthylalanine, 2-indanylglycine carboxylic acid, dihydrotryptophan, and benzoylphenylalanine. In certain embodiments of the invention X′aa is an analog of Trp that has increased hydrophobic character relative to Trp. For example, X′aa may be 1-methyltryptophan. In certain embodiments of the invention Xaa is an analog of Trp that has increased propensity to form hydrogen bonds relative to Trp but, in certain embodiments, not having increased hydrophobic character relative to Trp. In certain embodiments of the invention the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan.

In certain embodiments of the invention the peptide has a core sequence of X′aa-Gln-Asp-Xaa-Gly-X″aa (SEQ ID NO: 4), where X′aa and Xaa are each independently selected from Trp and analogs of Trp and X″aa is selected from His, Ala, analogs of Ala, Phe, and Trp. In certain embodiments of the invention X′aa is an analog of Trp that has increased hydrophobic character relative to Trp, such as 1-methyltryptophan or another Trp analog having an alkyl substituent on the indole ring (e.g., at position 1, 4, 5, or 6). In certain embodiments X′aa is an analog of Trp that comprises a substituted or unsubstituted bicyclic aromatic ring component or two or more substituted or unsubstituted monocyclic aromatic ring components. In certain embodiments of the invention X′aa is selected from the group consisting of 2-napthylalanine, 1-napthylalanine, 2-indanylglycine carboxylic acid, dihydrotryptophan, and benzoylphenylalanine. In certain embodiments of the invention Xaa is an analog of Trp that has increased propensity to form hydrogen bonds with C3 relative to Trp but, in certain embodiments, not having increased hydrophobic character relative to Trp. In certain embodiments of the invention the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan. In certain embodiments X″aa is Ala or an analog of Ala such as Abu or another unbranched single methyl amino acid. In certain embodiments of the invention the peptide has a core sequence of X′aa-Gln-Asp-Xaa-Gly-X″aa (SEQ ID NO: 4), where X′aa and Xaa are each independently selected from Trp, analogs of Trp, and amino acids or amino acid analogs comprising at least one aromatic side chain, and X″aa is selected from His, Ala, analogs of Ala, Phe, and Trp. In certain embodiments X″aa is selected from analogs of Trp, aromatic amino acids, and aromatic amino acid analogs.

In certain preferred embodiments of the invention the peptide is cyclic. The peptide may be cyclized via a bond between any two amino acids, one of which is (X′aa)ₙ and the other of which is located within (X″aa)ₘ. In certain embodiments the cyclic portion of the peptide is between 9 and 15 amino acids in length, e.g., 10-12 amino acids in length. In certain embodiments the cyclic portion of the peptide is 11 amino acids in length, with a bond (e.g., a disulfide bond) between amino acids at positions 2 and 12. For example, the peptide may be 13 amino acids long, with a bond between amino acids at positions 2 and 12 resulting in a cyclic portion 11 amino acids in length.

In certain embodiments the peptide comprises or consists of the sequence X′aa1-X′aa2-X′aa3-X′aa4-Gln-Asp-Xaa-Gly-X″aa1-X″aa2-X″aa3-X″aa4-X″aa5 (SEQ ID NO: 5). In certain embodiments X′aa4 and Xaa are selected from Trp and analogs of Trp, and X′aa1, X′aa2, X′aa3, X″aa1, X″aa2, X″aa3, X″aa4, and X″aa5 are independently selected from among amino acids and amino acid analogs. In certain embodiments X′aa4 and Xaa are selected from aromatic amino acids and aromatic amino acid analogs. Any one or more of X′aa1, X′aa2, X′aa3, X″aa1, X″aa2, X″aa3, X″aa4, and X″aa5 may be identical to the amino acid at the corresponding position in compstatin. In one embodiment, X″aa1 is Ala or a single methyl unbranched amino acid. The peptide may be cyclized via a covalent bond between (i) X′aa1, X′aa2, or X′aa3; and (ii) X″aa2, X″aa3, X″aa4 or X″aa5. In one embodiment the peptide is cyclized via a covalent bond between X′aa2 and X″aa4. In one embodiment the covalently bound amino acid are each Cys and the covalent bond is a disulfide (S—S) bond. In other embodiments the covalent bond is a C—C, C—O, C—S, or C—N bond. In certain embodiments one of the covalently bound residues is an amino acid or amino acid analog having a side chain that comprises a primary or secondary amine, the other covalently bound residue is an amino acid or amino acid analog having a side chain that comprises a carboxylic acid group, and the covalent bond is an amide bond. Amino acids or amino acid analogs having a side chain that comprises a primary or secondary amine include lysine and diaminocarboxylic acids of general structure NH₂(CH₂)ₙCH(NH₂)COOH such as 2,3-diaminopropionic acid (dapa), 2,4-diaminobutyric acid (daba), and ornithine (orn), wherein n=1 (dapa), 2 (daba), and 3 (orn), respectively. Examples of amino acids having a side chain that comprises a carboxylic acid group include dicarboxylic amino acids such as glutamic acid and aspartic acid. Analogs such as beta-hydroxy-L-glutamic acid may also be used.

In certain embodiments, the compstatin analog is a compound that comprises a peptide having a sequence: -Xaal-Cys-Val-Xaa2-Gln-Asp-Xaa2*-Gly-Xaa3-His-Arg-Cys-Xaa4 (SEQ ID NO: 6); wherein: Xaal is Ile, Val, Leu, B¹-Ile, B¹-Val, B¹-Leu or a dipeptide comprising Gly-Ile or B¹-Gly-Ile, and B¹ represents a first blocking moiety; Xaa2 and Xaa2* are independently selected from Trp and analogs of Trp; Xaa3 is His, Ala or an analog of Ala, Phe, Trp, or an analog of Trp; Xaa4 is L-Thr, D-Thr, Ile, Val, Gly, a dipeptide selected from Thr-Ala and Thr-Asn, or a tripeptide comprising Thr-Ala-Asn, wherein a carboxy terminal-OH of any of the L-Thr, D-Thr, Ile, Val, Gly, Ala, or Asn optionally is replaced by a second blocking moiety B²; and the two Cys residues are joined by a disulfide bond. In other embodiments Xaa1 is absent or is any amino acid or amino acid analog, and Xaa2, Xaa2*, Xaa3, and Xaa4 are as defined above. If Xaal is absent, the N-terminal Cys residue may have a blocking moiety B¹ attached thereto. In another embodiment, Xaa4 is any amino acid or amino acid analog and Xaa1, Xaa2, Xaa2*, and Xaa3 are as defined above. In another embodiment Xaa4 is a dipeptide selected from the group consisting of: Thr-Ala and Thr-Asn, wherein the carboxy terminal —OH or the Ala or Asn is optionally replaced by a second blocking moiety B².

In any of the embodiments of the compstatin analog of SEQ ID NO: 6, Xaa2 may be Trp. In any of the embodiments of the compstatin analog of SEQ ID NO: 6, Xaa2 may be an analog of Trp comprising a substituted or unsubstituted bicyclic aromatic ring component or two or more substituted or unsubstituted monocyclic aromatic ring components. For example, the analog of Trp may be selected from 2-naphthylalanine (2-NaI), 1-naphthylalanine (1-NaI), 2-indanylglycine carboxylic acid (Ig1), dihydrotrpytophan (Dht), and 4-benzoyl-L-phenylalanine. In any of the embodiments of the compstatin analog of SEQ ID NO: 6, Xaa2 may be an analog of Trp having increased hydrophobic character relative to Trp. For example, the analog of Trp may be selected from 1-methyltryptophan, 4-methyltryptophan, 5-methyltryptophan, and 6-methyltryptophan. In one embodiment, the analog of Trp is 1-methyltryptophan. In one embodiment, Xaa2 is 1-methyltryptophan, Xaa2* is Trp, Xaa3 is Ala, and the other amino acids are identical to those of compstatin. In any of the embodiments of the compstatin analog of SEQ ID NO: 6, Xaa2* may be an analog of Trp such as an analog of Trp having increased hydrogen bond forming propensity with C3 relative to Trp, which, in certain embodiments, does not have increased hydrophobic character relative to Trp. In certain embodiments the analog of Trp comprises an electronegative substituent on the indole ring. For example, the analog of Trp may be selected from 5-fluorotryptophan and 6-fluorotryptophan. In certain embodiments of the invention Xaa2 is Trp and Xaa2* is an analog of Trp having increased hydrogen bond forming propensity with C3 relative to Trp which, in certain embodiments, does not have increased hydrophobic character relative to Trp. In certain embodiments of the compstatin analog of SEQ ID NO: 6, Xaa2 is analog of Trp having increased hydrophobic character relative to Trp such as an analog of Trp selected from 1-methyltryptophan, 4-methyltryptophan, 5-methyltryptophan, and 6-methyltryptophan, and Xaa2* is an analog of Trp having increased hydrogen bond forming propensity with C3 relative to Trp which, in certain embodiments, does not have increased hydrophobic character relative to Trp. For example, in one embodiment Xaa2 is methyltryptophan and Xaa2* is 5-fluorotryptophan.

In certain of the afore-mentioned embodiments, Xaa3 is Ala. In certain of the afore-mentioned embodiments Xaa3 is a single methyl unbranched amino acid, e.g., Abu.

The invention further provides compstatin analogs of SEQ ID NO: 6, as described above, wherein Xaa2 and Xaa2* are independently selected from Trp, analogs of Trp, and other amino acids or amino acid analogs that comprise at least one aromatic ring, and Xaa3 is His, Ala or an analog of Ala, Phe, Trp, an analog of Trp, or another aromatic amino acid or aromatic amino acid analog.

In certain embodiments of the invention the blocking moiety present at the N- or C-terminus of any of the compstatin analogs described herein is any moiety that stabilizes a peptide against degradation that would otherwise occur in mammalian (e.g., human or non-human primate) blood or interstitial fluid. For example, blocking moiety B¹ could be any moiety that alters the structure of the N-terminus of a peptide so as to inhibit cleavage of a peptide bond between the N-terminal amino acid of the peptide and the adjacent amino acid. Blocking moiety B² could be any moiety that alters the structure of the C-terminus of a peptide so as to inhibit cleavage of a peptide bond between the C-terminal amino acid of the peptide and the adjacent amino acid. Any suitable blocking moieties known in the art could be used. In certain embodiments of the invention blocking moiety B¹ comprises an acyl group (i.e., the portion of a carboxylic acid that remains following removal of the —OH group, also referred to herein as “alkanoyl”). The acyl group typically comprises between 1 and 12 carbons, e.g., between 1 and 6 carbons. For example, in certain embodiments of the invention blocking moiety B¹ is selected from the group consisting of: formyl, acetyl, proprionyl, butyryl, isobutyryl, valeryl, isovaleryl, etc. In one embodiment, the blocking moiety B¹ is an acetyl group, i.e., Xaal is Ac-Ile, Ac-Val, Ac-Leu, or Ac-Gly-Ile.

In certain embodiments of the invention blocking moiety B² is a primary or secondary amine (—NH₂ or —NHR¹, wherein R is an organic moiety such as an alkyl group).

In certain embodiments of the invention blocking moiety B¹ is any moiety that neutralizes or reduces the negative charge that may otherwise be present at the N-terminus at physiological pH. In certain embodiments of the invention blocking moiety B² is any moiety that neutralizes or reduces the negative charge that may otherwise be present at the C-terminus at physiological pH.

In certain embodiments of the invention, the compstatin analog is acetylated or amidated at the N-terminus and/or C-terminus, respectively. A compstatin analog may be acetylated at the N-terminus, amidated at the C-terminus, and or both acetylated at the N-terminus and amidated at the C-terminus. In certain embodiments of the invention a compstatin analog comprises an alkyl or aryl group at the N-terminus rather than an acetyl group.

In certain embodiments, the compstatin analog is a compound that comprises a peptide having a sequence: Xaal-Cys-Val-Xaa2-Gln-Asp-Xaa2*-Gly-Xaa3-His-Arg-Cys-Xaa4 (SEQ ID NO: 7); wherein: Xaal is Ile, Val, Leu, Ac-Ile, Ac-Val, Ac-Leu or a dipeptide comprising Gly-Ile or Ac-Gly-Ile; Xaa2 and Xaa2* are independently selected from Trp and analogs of Trp; Xaa3 is His, Ala or an analog of Ala, Phe, Trp, or an analog of Trp; Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of L-Thr, D-Thr, Ile, Val, Gly, Ala, or Asn optionally is replaced by —NH₂; and the two Cys residues are joined by a disulfide bond. Xaal, Xaa2, Xaa2*, Xaa3, and Xaa4 are as described above for the various embodiments of SEQ ID NO: 6. For example, in certain embodiments Xaa2* is Trp. In certain embodiments Xaa2 is an analog of Trp having increased hydrophobic character relative to Trp, e.g., 1-methyltryptophan. In certain embodiments Xaa3 is Ala. In certain embodiments Xaa3 is a single methyl unbranched amino acid. In certain embodiments of the invention Xaa1 is Ile and Xaa4 is L-Thr. In certain embodiments of the invention Xaa1 is Ile, Xaa2* is Trp, and Xaa4 is L-Thr.

The invention further provides compstatin analogs of SEQ ID NO: 7, as described above, wherein Xaa2 and Xaa2* are independently selected from Trp, analogs of Trp, other amino acids or aromatic amino acid analogs, and Xaa3 is His, Ala or an analog of Ala, Phe, Trp, an analog of Trp, or another aromatic amino acid or aromatic amino acid analog.

In certain embodiments of any of the compstatin analogs described herein, an analog of Phe is used rather than Phe.

Table 1 provides a non-limiting list of compstatin analogs useful in various embodiments of the present invention. The analogs are referred to in abbreviated form in the left column by indicating specific modifications at designated positions (1-13) as compared to the parent peptide, compstatin (amidated at the C-terminus). Unless otherwise indicated, peptides are amidated at the C-terminus Bold text is used to indicate certain modifications. Activity relative to compstatin (in this case compstatin amidated at the C-terminus) is based on published data and assays described therein (WO2004/026328, Mallik, 2005; Katragadda, 2006). Where multiple publications reporting an activity were consulted, the more recently published value is used, and it will be recognized that values may be adjusted in the case of differences between assays. It will also be appreciated that in certain embodiments of the invention the peptides listed in Table 1 are cyclized via a disulfide bond between the two Cys residues when used in the therapeutic compositions and methods of the invention. Alternate means for cyclizing the peptides are also within the scope of the invention.

TABLE 1

| | | SEQ ID | Activity over |
|---|---|---|---|
| Peptide | Sequence | NO: | compstatin |
| Compstatin | H-ICVVQDWGHHRCT-CONH₂ | 8 | * |
| Ac-compstatin | Ac-ICVVQDWGHHRCT-CONH₂ | 9 | 3 × more |
| Ac-V4Y/H9A | Ac-ICVYQDWGAHRCT-CONH₂ | 10 | 14 × more |
| Ac-V4W/H9A —OH | Ac-ICVWQDWGAHRCT-COOH | 11 | 27 × more |
| Ac-V4W/H9A | Ac-ICVWQDWGAHRCT-CONH₂ | 12 | 45 × more |
| Ac-V4W/H9A/T13dT —OH | Ac-ICVWQDWGAHRCdT-COOH | 13 | 55 × more |
| Ac-V4(2-Nal)/H9A | Ac-ICV(2-Nal)QDWGAHRCT-CONH₂ | 14 | 99 × more |
| Ac V4(2-Nal)/H9A —OH | Ac-ICV(2-Nal)QDWGAHRCT-COOH | 15 | 38 × more |
| Ac V4(1-Nal)/H9A —OH | Ac-ICV(1-Nal)QDWGAHRCT-COOH | 16 | 30 × more |
| Ac-V42Igl/H9A | Ac-ICV(2-Igl)QDWGAHRCT-CONH₂ | 17 | 39 × more |
| Ac-V42Igl/H9A —OH | Ac-ICV(2-Igl)QDWGAHRCT-COOH | 18 | 37 × more |
| Ac-V4Dht/H9A —OH | Ac-ICVDhtQDWGAHRCT-COOH | 19 | 5 × more |
| Ac-V4(Bpa)/H9A —OH | Ac-ICV(Bpa)QDWGAHRCT-COOH | 20 | 49 × more |
| Ac-V4(Bpa)/H9A | Ac-ICV(Bpa)QDWGAHRCT-CONH₂ | 21 | 86 × more |
| Ac-V4(Bta)/H9A —OH | Ac-ICV(Bta)QDWGAHRCT-COOH | 22 | 65 × more |
| Ac-V4(Bta)/H9A | Ac-ICV(Bta)QDWGAHRCT-CONH₂ | 23 | 64 × more |
| Ac-V4W/H9(2-Abu) | Ac-ICVWQDWG(2-Abu)HRCT-CONH₂ | 24 | 64 × more |
| +G/V4W/H9A +AN —OH | H-GICVWQDWGAHRCTAN-COOH | 25 | 38 × more |
| Ac-V4(5fW)/H9A | Ac-ICV(5fW)QDWGAHRCT-CONH₂ | 26 | 31 × more |
| Ac-V4(5-MeW)/H9A | Ac-ICV(5-methyl-W)QDWGAHRCT-CONH₂ | 27 | 67 × more |
| Ac-V4(1MeW)/W7(5fW)/H9A | Ac-ICV(1-methyl-W)QD(5fW)GAHRCT-CONH₂ | 28 | 264 × more |
| Ac-V4W/W7(5fW)/H9A | Ac-ICVWQD(5fW)GAHRCT-CONH₂ | 29 | 121 × more |
| Ac-V4(5fW)/W7(5fW)/H9A | Ac-ICV(5fW)QD(5fW)GAHRCT-CONH₂ | 30 | 161 × more |
| Ac-V4(5-MeW)/W7(5fW)H9A | Ac-ICV(5-methyl-W)QD(5fW)GAHRCT-CONH₂ | 31 | NA |
| Ac-V4(1-MeW)/H9A | Ac-ICV(1-methyl-W)QDWGAHRCT-CONH₂ | 32 | 264 × more |
| +G/V4(6fW)/W7(6fW) | H-GICV(6fW)QD(6fW)GAHRCTN-COOH | 33 | 126 × more |
| H9A + N—OH | | | |
| Ac-V4(1-formyl-W)/H9A | Ac-ICV(1-formyl-W)QDWGAHRCT-CONH₂ | 34 | 264 × more |
| Ac-V4(5-methoxy-W)/H9A | Ac-ICV(1-methyoxy-W)QDWGAHRCT-CONH₂ | 35 | 76 × more |
| G/V4(5f-W)/W7(5fW)/ | H-GICV(5fW)QD(5fW)GAHRCTN-COOH | 36 | 112 × more |
| H9A + N—OH | | | |

NA = not available

In certain embodiments of the compositions and methods of the invention the compstatin analog has a sequence selected from sequences 9-36. In certain embodiments of the compositions and methods of the invention the compstatin analog has a sequence selected from SEQ ID NOs: 14, 21, 28, 29, 30, 32, 33, 34, or 36. In certain embodiments of the invention the compstatin analog has between 1 and 99-fold the activity of compstatin. In certain embodiments of the invention the compstatin analog has between 99 and 200-fold the activity of compstatin. In certain embodiments of the invention the compstatin analog has greater than 200-fold the activity of compstatin. In certain embodiments of the compositions and methods of the invention the compstatin analog has SEQ ID NO: 28. In certain embodiments of the compositions and methods of the invention the compstatin analog has SEQ ID NO: 29. In certain embodiments of the compositions and methods of the invention the compstatin analog has SEQ ID NO: 30. In certain embodiments of the compositions and methods of the invention the compstatin analog has SEQ ID NO: 32. In certain embodiments of the compositions and methods of the invention the compstatin analog has SEQ ID NO: 33. In certain embodiments of the compositions and methods of the invention the compstatin analog has SEQ ID NO: 34. In certain embodiments of the compositions and methods of the invention the compstatin analog has SEQ ID NO: 36.

In certain embodiments of the compositions and methods of the invention the compstatin analog has a sequence as set forth in Table 1, but where the Ac- group is replaced by an alternate blocking moiety B¹, as described above. In some embodiments the —NH₂ group is replaced by an alternate blocking moiety B², as described above.

In one embodiment, the compstatin analog binds to substantially the same region of the β chain of human C3 as does compstatin. In one embodiment the compstatin analog is a compound that binds to a fragment of the C-terminal portion of the β chain of human C3 having a molecular weight of about 40 kDa to which compstatin binds (Soulika, A. M., et al., Mol. Immunol., 35:160, 1998; Soulika, A. M., et al., Mol. Immunol. 43(12):2023-9, 2006). In certain embodiments the compstatin analog is a compound that binds to the binding site of compstatin as determined in a compstatin-C3 structure, e.g., a crystal structure or NMR-derived 3D structure. In certain embodiments the compstatin analog is a compound that could substitute for compstatin in a compstatin-C3 structure and would form substantially the same intermolecular contacts with C3 as compstatin. In certain embodiments the compstatin analog is a compound that binds to the binding site of a peptide having a sequence set forth in Table 1, e.g., SEQ ID NO: 28, 29, 30, 32, 33, 34, or 36 in a peptide-C3 structure, e.g., a crystal structure. In certain embodiments the compstatin analog is a compound that could substitute for the peptide of SEQ ID NO: 8-36, e.g., SEQ ID NO: 28, 29, 30, 32, 33, 34, or 36 in a peptide-C3 structure and would form substantially the same intermolecular contacts with C3 as the peptide.

One of ordinary skill in the art will readily be able to determine whether a compstatin analog binds to a fragment of the C-terminal portion of the β chain of C3 using routine experimental methods. For example, one of skill in the art could synthesize a photocrosslinkable version of the compstatin analog by including a photo-crosslinking amino acid such as p-benzoyl-L-phenylalanine (Bpa) in the compound, e.g., at the C-terminus of the sequence (Soulika, A. M., et al, supra). Optionally additional amino acids, e.g., an epitope tag such as a FLAG tag or an HA tag could be included to facilitate detection of the compound, e.g., by Western blotting. The compstatin analog is incubated with the fragment and crosslinking is initiated. Colocalization of the compstatin analog and the C3 fragment indicates binding. Surface plasmon resonance may also be used to determine whether a compstatin analog binds to the compstatin binding site on C3 or a fragment thereof. One of skill in the art would be able to use molecular modeling software programs to predict whether a compound would form substantially the same intermolecular contacts with C3 as would compstatin or a peptide having the sequence of any of the peptides in Table 1, e.g., SEQ ID NO: 28, 29, 30, 32, 33, 34, or 36. Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, and/or WO2004026328. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3^{rd} ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g. to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

Compstatin Mimetics

The structure of compstatin is known in the art, and NMR structures for a number of compstatin analogs having higher activity than compstatin are also known (Malik, supra). Structural information may be used to design compstatin mimetics.

In one embodiment, the compstatin mimetic is any compound that competes with compstatin or any compstatin analog (e.g., a compstatin analog whose sequence is set forth in Table 1) for binding to C3 or a fragment thereof (such as a 40 kD fragment of the β chain to which compstatin binds). In some embodiments, the compstatin mimetic has an activity equal to or greater than that of compstatin. In some embodiments, the compstatin mimetic is more stable, orally available, or has a better bioavailability than compstatin. The compstatin mimetic may be a peptide, nucleic acid, or small molecule. In certain embodiments the compstatin mimetic is a compound that binds to the binding site of compstatin as determined in a compstatin-C3 structure, e.g., a crystal structure or a 3-D structure derived from NMR experiments. In certain embodiments the compstatin mimetic is a compound that could substitute for compstatin in a compstatin-C3 structure and would form substantially the same intermolecular contacts with C3 as compstatin. In certain embodiments the compstatin mimetic is a compound that binds to the binding site of a peptide having a sequence set forth in Table 1, e.g., SEQ ID NO: 28, 29, 30, 32, 33, 34, or 36 in a peptide-C3 structure. In certain embodiments the compstatin mimetic is a compound that could substitute for a peptide having a sequence set forth in Table 1, e.g., SEQ ID NO: 28, 29, 30, 32, 33, 34, or 36 in a peptide-C3 structure and would form substantially the same intermolecular contacts with C3 as the peptide. In certain embodiments the compstatin mimetic has a non-peptide backbone but has side chains arranged in a sequence designed based on the sequence of compstatin.

One of skill in the art will appreciate that once a particular desired conformation of a short peptide has been ascertained, methods for designing a peptide or peptidomimetic to fit that conformation are well known. See, e.g., G. R. Marshall (1993), Tetrahedron, 49: 3547-3558; Hruby and Nikiforovich (1991), in Molecular Conformation and Biological Interactions, P. Balaram & S. Ramasehan, eds., Indian Acad. of Sci., Bangalore, P P. 429-455), Eguchi M, Kahn M., Mini Rev Med Chem., 2(5):447-62, 2002. Of particular relevance to the present invention, the design of peptide analogs may be further refined by considering the contribution of various side chains of amino acid residues, e.g., for the effect of functional groups or for steric considerations as described in the art for compstatin and analogs thereof, among others.

It will be appreciated by those of skill in the art that a peptide mimic may serve equally well as a peptide for the purpose of providing the specific backbone conformation and side chain functionalities required for binding to C3 and inhibiting complement activation. Accordingly, it is contemplated as being within the scope of the present invention to produce and utilize C3-binding, complement-inhibiting compounds through the use of either naturally-occurring amino acids, amino acid derivatives, analogs or non-amino acid molecules capable of being joined to form the appropriate backbone conformation. A non-peptide analog, or an analog comprising peptide and non-peptide components, is sometimes referred to herein as a “peptidomimetic” or “isosteric mimetic,” to designate substitutions or derivations of a peptide that possesses much the same backbone conformational features and/or other functionalities, so as to be sufficiently similar to the exemplified peptides to inhibit complement activation. More generally, a compstatin mimetic is any compound that would position pharmacophores similarly to their positioning in compstatin, even if the backbone differs.

The use of peptidomimetics for the development of high-affinity peptide analogs is well known in the art. Assuming rotational constraints similar to those of amino acid residues within a peptide, analogs comprising non-amino acid moieties may be analyzed, and their conformational motifs verified, by means of the Ramachandran plot (Hruby & Nikiforovich 1991), among other known techniques.

One of skill in the art will readily be able to establish suitable screening assays to identify additional compstatin mimetics and to select those having desired inhibitory activities. For example, compstatin or an analog thereof could be labeled (e.g., with a radioactive or fluorescent label) and contacted with C3 in the presence of different concentrations of a test compound. The ability of the test compound to diminish binding of the compstatin analog to C3 is evaluated. A test compound that significantly diminishes binding of the compstatin analog to C3 is a candidate compstatin mimetic. For example, a test compound that diminishes steady-state concentration of a compstatin analog-C3 complex, or that diminishes the rate of formation of a compstatin analog-C3 complex by at least 25%, or by at least 50%, is a candidate compstatin mimetic. One of skill in the art will recognize that a number of variations of this screening assay may be employed. Compounds to be screened include natural products, libraries of aptamers, phage display libraries, compound libraries synthesized using combinatorial chemistry, etc. The invention encompasses synthesizing a combinatorial library of compounds based upon the core sequence described above and screening the library to identify compstatin mimetics. Any of these methods could also be used to identify new compstatin analogs having higher inhibitory activity than compstatin analogs tested thus far.

Other Compounds that Inhibit C3Activation and/or C3bActivity

Other compounds, e.g., small molecules, antibodies (but see discussion below regarding antibodies), antibody fragments, polypeptides, peptides, aptamers, etc., that bind to C3 are of use in certain embodiments of the invention. In certain embodiments of the invention the complement activation inhibitor comprises an Efb protein or fragment thereof from Staphylococcus aureus (or similar microorganism) or a derivative of such protein or fragment that can bind to C3 and inhibit its activation and/or bind to and inhibit C3b. Exemplary agents are described in PCT Application Pub. WO/2004/094600. Aptamers that bind to and inhibit C3 may be identified using methods such as SELEX (discussed below). U.S. Pat. Pub. No. 20030191084 discloses aptamers that bind to C3.

Compounds that Inhibit Factor B Activity

In certain embodiments the complement inhibitor inhibits activity of factor B. For example, the complement inhibitor may bind to factor B. The compound may inhibit activation of factor B or may bind to activated factor B (e.g., subunit Bb) and inhibit its activity, thereby inhibiting cleavage of C3. Exemplary agents include antibodies (but see discussion below regarding antibodies), antibody fragments, polypeptides, peptides, small molecules, and aptamers. Exemplary antibodies that inhibit factor B are described in U.S. Pat. Pub. No. 20050260198 and/or U.S. Ser. No. 11/057,047. In certain embodiments the isolated antibody or antigen-binding fragment selectively binds to factor B within the third short consensus repeat (SCR) domain. In certain embodiments the antibody or antigen-binding fragment prevents formation of a C3bBb complex. In certain embodiments the antibody or antigen-binding fragment prevents or inhibits cleavage of factor B by factor D. In certain embodiments the complement inhibitor is an antibody, antibody fragment, small molecule, aptamer, or polypeptide that binds to substantially the same binding site on factor B as an antibody described in U.S. Pat. Pub. No. 20050260198 and/or U.S. Ser. No. 11/057,047. The invention specifically encompasses administering an antibody or antibody fragment to factor B within 1 hour or less, e.g., within less than 1 hour, e.g., within 5-30, 5-45, or 5-59 minutes after trauma. Use of peptides that bind to and inhibit factor B, which may be identified using methods such as phage display, is within the scope of the invention. Use of aptamers that bind to and inhibit factor B, which may be identified using methods such as SELEX, is within the scope of the invention.

Compounds that Inhibit Factor D Activity

In certain embodiments the complement inhibitor inhibits factor D. For example, the complement inhibitor may bind to factor D. Exemplary agents include antibodies (but see discussion below), antibody fragments, peptides, small molecules, and aptamers. Exemplary antibodies that inhibit factor D are described in U.S. Ser. No. 09/821,255 (U.S. Pat. Pub. No. 20020081293). In certain embodiments the complement inhibitor is an antibody, antibody fragment, small molecule, aptamer, or polypeptide that binds to substantially the same binding site on factor D as an antibody described in U.S. Pat. No. 7,112,327. Exemplary polypeptides that inhibit alternative pathway activation and are believed to inhibit factor D are disclosed in U.S. Pub. No. 20040038869. Use of peptides that bind to and inhibit factor D, which may be identified using methods such as phage display, is within the scope of the invention. Use of aptamers that bind to and inhibit factor D, which may be identified using methods such as SELEX, is within the scope of the invention.

Compounds that Inhibit Properdin

In some embodiments of the invention antiproperdin antibodies, antibody fragment, or other anti-properdin agents are used. See, e.g., U.S. Pat. Pub. No. 20030198636.

Compounds that Inhibit Components of Lectin Pathway

In some embodiments the compounds inhibit one or more components of the lectin pathway. See, e.g., WO/2007/117996) METHODS FOR TREATING CONDITIONS ASSOCIATED WITH MASP-2 DEPENDENT COMPLEMENT ACTIVATION.

Multimodal Complement Inhibitors

In certain embodiments of the invention the complement inhibitor binds to more than one complement protein and/or inhibits more than one step in a complement activation pathway. Such complement inhibitors are referred to herein as “multimodal”. One aspect of this invention is the recognition of the advantages of inhibiting complement activation at multiple points in the complement activation pathway in a trauma patient by administration of a multimodal complement inhibitor. In certain embodiments of the invention the complement inhibitor is a virus complement control protein (VCCP). The invention specifically contemplates use of any of the agents described in U.S. Ser. No. 11/247,886 and PCT/US2005/36547, filed Oct. 8, 2005. Poxviruses and herpesviruses are families of large, complex viruses with a linear double-stranded DNA genome. Certain of these viruses encode immunomodulatory proteins that are believed to play a role in pathogenesis by subverting one or more aspects of the normal immune response and/or fostering development of a more favorable environment in the host organism (Kotwal, G J, Immunology Today, 21(5), 242-248, 2000). Among these are VCCPs. Poxvirus complement control proteins are members of the complement control protein (CCP) superfamily and typically contain 4 SCR modules. These proteins have features that make them advantageous for complement inhibition in trauma victims accordance with the present invention. In certain embodiments the VCCP is a poxvirus complement control protein (PVCCP). The PVCCP can comprise a sequence encoded by, e.g., vaccinia virus, variola major virus, variola minor virus, cowpox virus, monkeypox virus, ectromelia virus, rabbitpox virus, myxoma virus, Yaba-like disease virus, or swinepox virus. In other embodiments the VCCP is a herpesvirus complement control protein (HVCCP). The HVCCP can comprise a sequence encoded by a Macaca fuscata rhadinovirus, cercopithecine herpesvirus 17, or human herpes virus 8. In other embodiments the HVCCP comprises a sequence encoded by herpes simplex virus saimiri ORF 4 or ORF 15 (Albrecht, J C. & Fleckenstein, B., J. Virol., 66, 3937-3940, 1992; Albrecht, J., et al., Virology, 190, 527-530, 1992).

The VCCP may inhibit the classical complement pathway, the alternate complement pathway, the lectin pathway, or any combination. In certain embodiments of the invention the VCCP, e.g., a PVCCP, binds to C3b, C4b, or both. In certain embodiments of the invention the PVCCP comprises one or more putative heparin binding sites (K/R-X-K/R) and/or possesses an overall positive charge. In some embodiments the PVCCP comprises at least 3 SCR modules (e.g., modules 1-3), e.g., 4 SCR modules. The PVCCP protein can be a precursor of a mature PVCCP (i.e., can include a signal sequence that is normally cleaved off when the protein is expressed in virus-infected cells) or a mature form (i.e., lacking the signal sequence).

VCP is described in U.S. Pat. Nos. 5,157,110 and 6,140,472, and in Kotwal, G K, et al., Nature, 355, 176-178, 1988. FIGS. 3A and 3B of U.S. Ser. No. 11/247,886 and PCT/US2005/36547 (WO2006042252) show the sequence of the precursor and mature VCP proteins, respectively. VCP blocks complement activation at multiple steps and reduces levels of the proinflammatory chemotactic factors C3a, C4a, and C5a.

Variola virus major and minor encode proteins that are highly homologous to VCP and are referred to as smallpox inhibitor of complement enzymes (SPICE) (Rosengard, A M, et al., Proc. Natl. Acad. Sci., 99(13), 8803-8813. U.S. Pat. No. 6,551,595). SPICE from various variola strains sequenced to date differs from VCP by about 5% (e.g., about 11 amino acid differences). Similarly to VCP, SPICE binds to C3b and C4b and causes their degradation, acting as a cofactor for factor I. However, SPICE degrades C3b approximately 100 times as fast as VCP and degrades C4b approximately 6 times as fast as VCP. The amino acid sequence of SPICE is presented in FIG. 6 (SEQ ID NO: 12) of U.S. Ser. No. 11/247,886 and PCT/U52005/36547 (WO2006042252)

It will be appreciated that the exact sequence of complement control proteins identified in different virus isolates may differ slightly. Use of such proteins falsl within the scope of the present invention. Complement control proteins from any such isolate may be used, provided that the protein has not undergone a mutation that substantially abolishes its activity. Thus the sequence of a VCCP such as SPICE or VCP may differ from the exact sequences presented herein or under the accession numbers listed in Table 2. It will also be appreciated that a number of amino acid alterations, e.g., additions, deletions, or substitutions such as conservative amino acid substitutions, may be made in a typical polypeptide such as a VCCP without significantly affecting its activity, such that the resulting protein is considered equivalent to the original polypeptide. In some embodiments the VCCP is at least 90%, 95%, 98%, or 99% identical in sequence to a sequence identified by accession number in Table 2.

TABLE 2
Representative Viral Complement Control Proteins

| Virus | Protein | Accession | Virus Type |
|---|---|---|---|
| Variola | D12L | NP_042056 | Orthopoxvirus |
| | D15L (SPICE) | AAA69423 | Orthopoxvirus |
| Vaccinia | VCP | AAO89304 | Orthopoxvirus |
| Cowpox | CPXV034 | AAM13481 | Orthopoxvirus |
| | C17L | CAA64102 | Orthopoxvirus |
| Monkeypox | D14L | AAV84857 | Orthopoxvirus |
| Ectromelia virus | Complement control | CAE00484 | Orthopoxvirus |
| | protein | | |
| Rabbitpox | RPXV017 | AAS49730 | Orthopoxvirus |
| Macaca fuscata | JM4 | AAS99981 | Rhadinavirus |
| rhadinovirus | | | (Herpesvirus) |
| Cercopithecine | Complement binding | NP_570746 | Herpesvirus |
| herpesvirus 17 | protein (ORF4) | | |
| Human herpes | Complement binding | AAB62602 | Herpesvirus |
| virus 8 | protein (ORF4) | | |

Mammalian Complement Regulatory Proteins

In some embodiments the complement inhibitor comprises at least a portion of a mammalian, e.g., human, complement regulatory protein such as CFH, CFI, CR1, DAF, MCP, CD59, C4 bp, and complement receptor 2 inhibitor trispanning (CRIT; Inal, J., et al, J Immunol., 174(1):356-66, 2005). In some embodiments of the invention the complement regulatory polypeptide is one that is normally membrane-bound in its naturally occurring state. In some embodiments of the invention a fragment of such polypeptide that lacks some or all of a transmembrane and/or intracellular domain is used. Soluble forms of complement receptor 1 (sCR1), for example, are of use in the invention. For example the compounds known as TP10 or TP20 (Avant Therapeutics) can be used. In some embodiments a soluble complement control protein, e.g., CFH, is used. In some embodiments the complement inhibitor is a C3b/C4b Complement Receptor-like molecule such as those described in U.S. Pat. Pub. No. 20020192758.

In certain embodiments of the invention the complement inhibitor comprises a chimeric polypeptide comprising a first polypeptide which inhibits complement activation, linked, e.g., covalently to a second polypeptide which inhibits complement activation, wherein the first and second polypeptides each comprise at least a portion of a mammalian complement regulatory protein. In some embodiments complement inhibitor comprises at least a portion of DAF and at least a portion of MCP. Exemplary chimeric polypeptides are disclosed, e.g., in U.S. Pat. No. 5,679,546, e.g., CAB-2 (also known as MLN-2222). In some embodiments the polypeptide comprises at least 4 SCR domains of at least one mammalian complement regulatory protein. In some embodiments the polypeptide comprises at least 4 SCR domains of each of first and second distinct mammalian complement regulatory proteins.

It will be appreciated that normal human blood contains certain regulatory proteins and human cells comprise complement regulatory proteins. The inventive methods do not consist merely of administering human blood, blood products (e.g., plasma, platelets, cryoprecipitate), and/or cells containing such complement inhibitors in amounts that would constitute conventional or otherwise previously used therapy for trauma. Of course human blood, blood products, and/or cells could be supplemented with a complement inhibitor such as those described herein. Such supplemented compositions are an aspect of the invention.

Compounds that Inhibit C5 Cleavage

In certain embodiments of the invention, a complement inhibitor that inhibits cleavage and activation of C5 is administered in combination with a complement activation inhibitor that acts at or above the level of C3 activation. For example, the complement inhibitor may bind to C5. Exemplary agents include antibodies, antibody fragments, polypeptides, small molecules, and aptamers. Exemplary antibodies are described in U.S. Pat. No. 6,534,058. Exemplary compounds that bind to and inhibit C5 are described in U.S. Pat. Pub. Nos. 20050090448 and 20060115476. In certain embodiments the complement inhibitor is an antibody, antibody fragment, small molecule, aptamer, or polypeptide that binds to substantially the same binding site on C5 as an antibody described in U.S. Pat. No. 6,534,058 or a peptide described in U.S. Ser. No. 10/937,912. U.S. Pat. Pub. No. 20060105980 discloses aptamers that bind to and inhibit C5.

Complement Inhibiting Antibodies

In certain embodiments of the invention the complement inhibitor is an antibody. Antibodies useful in certain embodiments of the invention could be of various species, e.g., human, rodent, rabbit, goat, chicken, and/or classes, e.g., the human classes: IgG (e.g., IgG1, IgG2, IgG3, IgG4), IgM, IgA, IgD, and IgE. However, complement inhibiting antibodies of use in the invention should have appropriate properties for use in trauma patients, as described above. Some complement inhibiting antibodies have a half-life that is too long to be suitable for use in the present invention and/or cause an irreversible alteration in structure of their target such that a complement component released from a complex with such an antibody would be substantially inactivatable. Administration of such antibodies would result in considerable levels of complement inhibition at time points greater than 24 or 48 hours post-injury and/or post-administration. However, antibody fragments typically have considerably shorter half-lives than conventional full size antibodies containing two light and two heavy chains. Accordingly, in various embodiments of the invention a complement inhibiting antibody fragment, such as an Fab′, F(ab′)₂, scFv (single-chain variable) or other fragment that retains an antigen binding site, or a recombinantly produced scFv fragment, is used. See, e.g., Allen, T., Nature Reviews Cancer, Vol. 2, 750-765, 2002, and references therein. It is specifically envisioned to generate fragments of antibodies known in the art as being able to inhibit complement activation or to use antigen-binding portions of such antibodies in certain of the inventive methods.

The antibody may be a chimeric or “humanized” antibody in which, for example, a variable domain of rodent origin is fused to a constant domain of human origin, or in which some or all of the complementarity-determining region amino acids often along with one or more framework amino acids are “grafted” from a rodent, e.g., murine, antibody to a human antibody, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16: 535-539. The antibody may be partially or completely humanized. An antibody may be polyclonal or monoclonal, though for purposes of the present invention monoclonal antibodies are generally preferred. Methods for generating antibodies that specifically bind to virtually any molecule of interest are known in the art. For example, monoclonal or polyclonal antibodies can be purified from natural sources, e.g., from blood or ascites fluid of an animal that produces the antibody (e.g., following immunization with the molecule or an antigenic fragment thereof) or can be produced recombinantly, in cell culture and, e.g., purified from culture medium. Suitable antibodies can be identified using phage display and related techniques. Affinity purification may be used, e.g., protein A/G affinity purification and/or affinity purification using the antigen as an affinity reagent. See, e.g., Kaser, M. and Howard, G., “Making and Using Antibodies: A Practical Handbook” and Sidhu, S., “Phage Display in Biotechnology and Drug Discovery”, CRC Press, Taylor and Francis Group, 2005, for further information.

F(ab′)₂ fragments can be generated, for example, through the use of an Immunopure F(ab′)₂ Preparation Kit (Pierce) in which the antibodies are digested using immobilized pepsin and purified over an immobilized Protein A column. The digestion conditions (such as temperature and duration) may be optimized by one of ordinary skill in the art to obtain a good yield of F(ab′)₂. The yield of F(ab′)₂ resulting from the digestion can be monitored by standard protein gel electrophoresis. F(ab′) can be obtained by papain digestion of antibodies, or by reducing the S—S bond in the F(ab′)₂.

Combination Complement Inhibitor Therapy

Combination therapy using two or more complement inhibitors is within the scope of the invention. The two or more complement inhibitors may be provided in the same composition or separately. In one embodiment at least two of the complement inhibitors are peptides, each having a length between 5 and 50 amino acids. Optionally, at least one of the peptides is cyclic. In certain embodiments the complement inhibitors bind to two or more different complement components. In certain embodiments the complement inhibitors bind to two or more different soluble complement proteins. In certain embodiments the complement inhibitors inhibit activation or activity of at least two complement proteins selected from C3, C5, factor B, and factor D. In certain embodiments a first complement inhibitor inhibits activation or activity of C3 and a second complement inhibitor inhibit activation or activity of a complement protein selected from C5, factor B, and factor D. In certain embodiments a first complement inhibitor inhibits activation or activity of C3 and a second complement inhibitor inhibit activation or activity of a complement protein selected from factor B and factor D. In certain embodiments a first complement inhibitor inhibits activation or activity of C3 and a second complement inhibitor inhibit activation or activity of a complement protein selected from C5 and C5a. For example, in certain embodiments a first complement inhibitor inhibits activation or activity of C3 and a second complement inhibitor binds to and inhibits C5aR. For example, cyclic peptides that inhibit C5aR may be used. Exemplary C5a receptor antagonists include a variety of small cyclic peptides such as those described in U.S. Pat. No. 6,821,950; U.S. Ser. No. 11/375,587; and/or PCT/US06/08960 (WO2006/099330), or a peptidomimetic thereof. In certain embodiments a cyclic peptide comprising the sequence [OPdChaWR] (SEQ ID NO: 39) is used. In certain embodiments a peptide comprising the sequence Xaa[OPdChaWR] (SEQ ID NO: 40) is used wherein [ ] denotes cyclization. Examples include CM-[OPdChaWR] (SEQ ID NO: 41), HCin-[OPdChaWR] (SEQ ID NO: 42), F-[OPdChaWR] (SEQ ID NO: 43), and F-[KPdChaWR] (SEQ ID NO: 44). In certain embodiments of the invention a first complement inhibitor binds to C3 and a second complement inhibitor is a complement regulatory protein that contributes to dissociation, inactivation or degradation of a convertase or activated complement component. Administration in combination means that the two or more agents are administered such that both are active in the body during time periods that at least partly overlap. They may be administered separately admininstered for example, within up to 5, 10, 30, or 60 minutes apart, or in some embodiments up to 1, 2, 4, 6, or 12 hours apart.

Targeting Complement Inhibitors to Sites of Injury

The invention provides a composition comprising (i) a complement inhibitor suitable for administration to a trauma patient (such as those described herein); and (ii) a binding moiety that binds to a biomolecule present at increased levels in injured tissue. In certain embodiments of the invention the binding moiety and the complement inhibitor are linked. The linkage can be covalent or noncovalent and can be direct or indirect. The binding moiety can be, for example, an antibody or ligand that binds to the biomolecule. According to certain embodiments of the invention the biomolecule is a cellular marker expressed on or at the surface of a cell of an individual who has suffered a traumatic injury at higher levels than would be found in the absence of the injury. In some embodiments the marker is a neoantigen present on or at the surface of cells that have been subjected to ischemia. In some embodiments the binding moiety comprises CR2 or a fragment thereof.

Biomolecules of interest can be any molecule present on or at the surface of a cell or noncellular molecular entity, wherein the biomolecule is present at increased levels in injured tissue. By “on or at the surface of the cell or noncellular molecular entity” is meant that the biomolecule is accessible to molecules present in the extracellular environment so that it can be recognized and bound by the moiety. The biomolecule may be entirely extracellular. The biomolecule may be inserted into the cell membrane. In certain embodiments of the invention the biomolecule may be partly or entirely within the membrane. As long as a sufficient portion of the biomolecule is exposed or accessible so that it can be recognized and bound, it will be said to be present on or at the surface. The marker could be a proteoglycan or portion thereof. The marker could be a molecule not normally found on the inner lining of blood vessel walls.

In various embodiments of the invention an appropriate binding moiety is a molecule that specifically binds to a target biomolecule through a mechanism other than an antigen-antibody interaction. Such a binding moiety is referred to as a “ligand”. For example, in various embodiments of the invention a ligand can be a polypeptide, peptide, nucleic acid (e.g., DNA or RNA), carbohydrate, lipid or phospholipid, or small molecule (e.g., an organic compound, whether naturally-occurring or artificially created that has relatively low molecular weight and is not a protein, polypeptide, nucleic acid, or lipid, typically with a molecular weight of less than about 1500 g/mol and typically having multiple carbon-carbon bonds).

Ligands may be naturally occurring or synthesized, including molecules whose structure has been invented by man. Examples of ligands include, but are not limited to, hormones, growth factors, or neurotransmitters that bind to particular receptors. It will also be appreciated that altered versions of the above-mentioned polypeptide ligands differing in sequence from their naturally occurring counterparts but retaining the ability to bind to the marker can also be used. In certain embodiments of the invention, a polypeptide ligand contains 5 or fewer amino acid differences, 10 or fewer amino acid differences, 25 or fewer amino acid differences, 50 or fewer amino acid differences, or 100 or fewer amino acid differences with respect to its naturally occurring counterpart. In certain embodiments of the invention the number of amino acid differences between a naturally occurring polypeptide ligand and a fragment or altered version thereof for use in the invention is 5% or less, 10% or less, or 25% or less of the total number of amino acids in the naturally occurring polypeptide.

In certain embodiments of the invention the ligand is an aptamer that binds to a cell type specific marker. In general, an aptamer is an oligonucleotide (e.g., DNA or RNA or) that binds to a particular protein. Aptamers are typically derived from an in vitro evolution process called SELEX, and methods for obtaining aptamers specific for a protein of interest are known in the art. See, e.g., Brody E N, Gold L. J Biotechnol., 74(1):5-13, 2000.

Small molecules can also be used as ligands. Methods for identifying such ligands are known in the art. For example in vitro screening of small molecule libraries, including combinatorial libraries, and computer-based screening, e.g., to identify small organic compounds that bind to concave surfaces (pockets) of proteins, can identify small molecule ligands for numerous proteins of interest (Huang, Z., Pharm. & Ther. 86: 201-215, 2000).

In certain embodiments of the invention binding moieties are not proteins or molecules that are typically used as carriers and conjugated to antigens for the purpose of raising antibodies. Examples are carrier proteins or molecules such as bovine serum albumin, keyhole limpet hemocyanin, bovine gamma globulin, and diphtheria toxin. In certain embodiments of the invention the cell binding moiety is not an Fc portion of an immunoglobulin molecule.

Methods for covalently or noncovalently linking moieties are known in the art and are described in U.S. Ser. No. 10/923,940. General methods for conjugation and cross-linking are described in “Cross-Linking”, Pierce Chemical Technical Library, available at the Web site having URL www.piercenet.com and originally published in the 1994-95 Pierce Catalog and references cited therein, in Wong S S, Chemistry of Protein Conjugation and Crosslinking, CRC Press Publishers, Boca Raton, 1991; and G. T. Hermanson, supra. See also, Allen, T. M., Nature Reviews Cancer, 2, 750-763, 2002, which describes methods of making targeted therapeutic agents. For example, according to certain embodiments of the invention a bifunctional crosslinking reagent is used to couple a compstatin analog with an antibody or ligand. In general, bifunctional crosslinking reagents contain two reactive groups, thereby providing a means of covalently linking two target groups. The reactive groups in a chemical crosslinking reagent typically belong to various classes including succinimidyl esters, maleimides, pyridyldisulfides, and iodoacetamides. Bifunctional chelating agents may also be used. Alternately, two or more polypeptide moieties can be produced as a fusion protein.

Complement Repletion and Enhancing Therapy

The invention provides the recognition that repleting and/or enhancing potential complement activity at certain times during and/or after the immediate post-trauma period, e.g., at least 4 hours after injury, e.g., at least 8, 12, or 24 hours after injury, e.g., at least 48 hours, e.g., at least 72 hours, after injury, is of use to reduce trauma-associated morbidity and mortality. “Replete” refers to supplementing complement activation capacity, e.g., to a level at least 25%, at least 50%, or at least 75% of normal, or to approximately normal. “Enhance” refers to supplementing complement activation capacity to a level greater than normal. In some embodiments of particular interest a composition that repletes and/or enhances potential complement activity (a “pro-repletion therapy”) is administered to an individual who has suffered a traumatic injury between 4 hours and 4 days after occurrence of the injury. The invention provides compositions and methods for such use. These aspects of the invention may be used together with administering a complement activation inhibitor in the immediate post-trauma period as described elsewhere herein, i.e., both aspects may be used to treat a trauma patient. However, they constitute aspects of the invention that may be used independently of the other inventive methods described herein. The invention provides each of the methods in this section used together with any one or more of the methods provided above.

The invention provides a composition comprising a complement component and a pharmaceutically acceptable carrier, e.g. a fluid suitable for IV administration. Such fluid may be one suitable for administration to a patient after trauma. The complement component may be selected from: C1, C2, C3, C4, C5, C6, C7, C8, C9, fB, fD, properdin, or a subunit or active fragment of any of the foregoing. In some embodiments the component is C3. In some embodiments the fragment is C3b. In some embodiments the component is C5. In some embodiments the component is above the level of C3 in a complement activation pathway (e.g., C1, C2, C4, fB, fD). In some embodiments multiple components are administered. In some embodiments multiple alternative pathway components are administered. In some embodiments multiple classical pathway components are administered.

Complement components may be produced using recombinant methods, chemical synthesis, purification from natural sources, etc. One of skill in the art will readily find the sequences of complement components, e.g., human sequences, in public databases and the literature and be able to employ standard methods for producing biological macromolecules. See, e.g., Schwaeble W, Immunobiology, 188(3):221-32, 1993, with respect to recombinant human fB and Sunghee, K., et al, J. Biol. Chem. 270 (41): 24399, 1995, with respect to recombinant human fD. It will be appreciated that multiple polymorphic variants of certain of these complement components exist. In some embodiments, the most common variant is administered. In some embodiments a less common variant is administered. In some embodiments a variant identical to that expressed by the individual is administered. In some embodiments an altered version at least 90%, 95%, 96%, 97%, 98%, or 99% identical to the most common polymorphic variant is administered, wherein such altered version has at least 80% of the activity of the naturally occurring molecule. In some embodiments a naturally occurring variant having higher activity relative to other variants is selected. See, e.g., Montes T, et al., Proc Natl Acad Sci USA.106(11):4366-71, 2009. In some embodiments a non-naturally occurring variant having higher activity than the most common variant is selected. See, e.g., Sunghee, K., et al., supra.

Also provided are compositions comprising a complement component, e.g., one of the foregoing, wherein at least some of the complement component is complexed with an agent that stabilizes the complement component, e.g., against proteolytic cleavage. The agent may be a complement activation inhibitor such as those described herein. The agent may be an antibody fragment, small molecule ligand, aptamer, or peptide. The agent may increase the biological half-life of the complement component. The agent may occlude or sterically hinder access by a convertase. The agent may be an allosteric regulator. In exemplary embodiments the complement component is C3 and the agent is optionally a compstatin analog. The compstatin analog may be one of the less potent analogs or one or the more potent analogs in various embodiments of the invention. In exemplary embodiments the complement component is C5 and the agent is optionally a peptide. In some embodiments at least two complement component complexes are administered.

The complex is formed in vitro, for example, by allowing the complement component and agent to contact each other in solution for a suitable time period. The complex may be purified from the solution and/or uncomplexed agent and/or complement component removed. The average half-life of the complex (e.g., in the solution or in plasma) may be, e.g., between 1 and 24 hours or any intervening range. For example the average half-life may be about 1, 2, 4, 6, 8, 12, 16, 20, or 24 hours. An important aspect of this inventive approach is that the complement component when released from the complex, is activatable, i.e., it is not irreversibly inhibited as a result of complex formation.

The complement component (in complexed form or not complexed) is administered one or more times to replete and/or enhance complement activation capacity of the subject. In some embodiments the complement component is administered once or more between 4 hours and 4 days following occurrence of trauma. In some embodiments of the invention the complement component is administered at one or more time points more than 24 hours following trauma, e.g., at least 48 hours following trauma, e.g., between 48 hours and 2, 4, 6 weeks after trauma.

In some embodiments the agent inhibits a soluble complement regulatory protein such as CFH, CFI, or C4BP. Such agents as are of use in the invention inhibit the inhibitory activity of such endogenous molecules, thereby repleting and/or enhancing complement activation capacity of the subject. In some embodiments the antibodies are humanized. The agents may specifically bind to one or more forms (e.g., different genetically encoded variants or splice variants) of the endogenous protein. Antibodies that bind to multiple genetically encoded variants of CHF may be used. For example, antibodies that bind to the form of CFH having Tyr at position 402 and to the form of CFH having His at position 402 may be used. Antibodies specific to the form of CFH having Tyr at position 402 may be used. In some embodiments, an anti-CFH antibody is administered to an individual who expresses the Tyr402 variant. In some embodiments the individual is homozygous for an allele that encodes CFH having Tyr at position 402. In some embodiments the individual is heterozygous for an allele that encodes CFH having Tyr at position 402. In some embodiments, an anti-CFH antibody is administered to an individual who does not express the His402 variant.

Antibodies that bind to CFH are known in the art. One example is the mAb known as 5H5 (Pinter, C., et al. AIDS Res. Hum. Retroviruses. 5:577-588). Other examples are mAbs produced by clones L20/3 and C18/3 (Hycult Biotechnology, b.v., VWR, etc.), clones OX-23 and 10-10 (AbDSerotec) See also, Alsenz, J., et al., Biochem J.; 232(3):841-50, 1985. These antibodies may be humanized using standard methods. Certain auto-antibodies that bind to CFH and compromise its complement inhibiting ability are found in individuals with atypical hemolytic uremic syndrome (see, e.g., Jozsi, M., et al., Blood, 110(5):1516-8, 2007). In some embodiments an anti-CFH antibody binds to substantially the same portion of CFH as a CFH autoantibody present in a subject with atypical hemolytic uremic syndrome. Also of use are agents that inhibit synthesis of such proteins, e.g. via RNA intereference, such as small interfering RNAs. In some embodiments an inhibitor of activity or synthesis of a complement regulatory protein is administered once or more between 4 and 48 hours after trauma. In some embodiments such an agent is admininistered once or more more than 24 hours after trauma, e.g., at least 48, 72, or more hours following trauma.

Administration of CFH Polypeptides

The invention provides a method of treating an individual who has suffered a traumatic injury comprising administering a CFH polypeptide or a CFH modulator to the individual. A CFH modulator is an agent that increases or enhances one or more activities of CFH, e.g., an activity that contributes to the ability of CFH to inhibit complement activation. The CFH modulator may be, e.g., a small molecule, nucleic acid, or a polypeptide. In some embodiments a CFH modulator is an anti-microRNA (anti-miRNA), wherein the anti-miRNA binds to an endogenous miRNA such as miR-146a that inhibits expression of CFH. The invention also provides similar methods of treating a subject who has suffered a traumatic injury comprising administering a CFHR1, CFHR2, CFHR3, CFHR4 or CFHR5 polypeptide to an individual. An anti-microRNA often comprises an oligonucleotide sufficiently complementary to a target miRNA such that the anti-microRNA hybridizes to the miRNA and inhibits its activity. In some embodiments the anti-miRNA comprises a sequence at least 90% identical, e.g., 100% identical to the target miRNA over at least 15, 16, 17, 18, 19, 20, 21, 22, or 23 consecutive nucleotides. In some embodiments an anti-microRNA is an antagomir. In some embodiments the anti-microRNA comprises an oligonucleotide conjugated to a lipid, e.g., cholesterol. Often the anti-microRNA comprises modified nucleosides and/or internucleoside linkages that reduce its susceptibility to degradation in vivo.

“CFH polypeptide” refers to a polypeptide whose sequence comprises a sequence identical to that of naturally occurring CFH (e.g., human CFH) or an active altered version thereof. An active altered version may have any one or more of the following activities of CFH: (1) binding to C-reactive protein (CRP); (2) binding to cell surfaces; (3) binding to heparin; (4) binding to various polyanions such as sialic acid and glycosaminoglycan chains of proteoglycans (which binding may at least in part mediate binding of CFH to cell surfaces); (5) binding to C3b; (6) reducing formation and activity of the alternative pathway C3-convertase (C3bBb); (7) accelerating decay of C3bBb; (8) acting as a cofactor for the serine protease CFI in the degradation of C3b; and (9) binding to adrenomedullin. In some embodiments the activity of an altered version as a complement inhibitor is at least 50%, 75%, 90% or more of the activity of CFH. “CFHR polypeptide” refers to a polypeptide whose sequence comprises a sequence identical to that of naturally occurring CFHR (e.g., human CFH) or an active altered version thereof.

A CFH polypeptide or CFHR polypeptide may be produced using recombinant nucleic acid technology in suitable host cells or purified from natural sources, e.g., plasma. See, e.g., PCT/EP2008/002238, Sim R B and DiScipio R G. Biochem J., 205:285-93, 1982, and Quiang, X., et al., Mol Med., 14(7-8):443-50, 2008, for descriptions of methods that may be used to purify CFH from human plasma. CFH, CFHR1, CFHR2, CFHR3, CFHR4 and CFHR5 sequences are readily available and may be used for recombinant expression. One of skill in the art can readily retrieve the sequences of these proteins, the sequences of the corresponding cDNAs and genomic DNA sequences, genomic sequences near such genes, and other information relating to the genes and proteins from publicly available databases such as those available via Entrez Global Query Cross-Database Search System (“Entrez”) available at the web site having URL www.ncbi.nlm.nih.gov/sites/entrez. GenBank may contain sequences of multiple variants of CFH, CFH-like proteins, and corresponding cDNAs. One of skill in the art may utilize the Reference Sequence (RefSeq) database, which provides one example of each natural biological molecule for major organisms and is incorporated herein by reference. The complete CFH cDNA and derived amino acid sequences were described by Ripoche et al. (Ripoche, J., et. al., Biochem J. 249:593-602, 1988). See also GenBank accession numbers NP_{—}000177.2 and Y00716. It will be appreciated that some protein sequence variants contain a tyrosine (Tyr; Y) while others contain histidine (His; H) at amino acid position 402 (position 384 of the mature CFH protein), and thus corresponding nucleic acid sequences differ, e.g., containing a T or C at nucleotide position 1277. There is an alternatively spliced form of CFH, known as FHL-1. The first 445 amino acids of CFH and FHL1 are identical, with FHL1 having a unique C-terminal 4 amino acids (exon 10A). The cDNA and amino acid sequences for FHL1 are present in GenBank and are incorporated herein by reference.

Exemplary CFH polypeptide sequences are shown below wherein the forms having tyrosine and histidine at position 402 are indicated.

(SEQ ID NO: 37)
MRLLAKIICLMLWAICVAEDCNELPPRRNTEILTGSWSDQTYPEGTQAIYKCRPGYRSL
GNVIMVCRKGEWVALNPLRKCQKRPCGHPGDTPFGTFTLTGGNVFEYGVKAVYTCN
EGYQLLGEINYRECDTDGWTNDIPICEVVKCLPVTAPENGKIVSSAMEPDREYHFGQA
VRFVCNSGYKIEGDEEMHCSDDGFWSKEKPKCVEISCKSPDVINGSPISQKIIYKENERF
QYKCNMGYEYSERGDAVCTESGWRPLPSCEEKSCDNPYIPNGDYSPLRIKHRTGDEIT
YQCRNGFYPATRGNTAKCTSTGWIPAPRCTLKPCDYPDIKHGGLYHENMRRPYFPVA
VGKYYSYYCDEHFETPSGSYWDHIHCTQDGWSPAVPCLRKCYFPYLENGYNQNYGR
KFVQGKSIDVACHPGYALPKAQTTVTCMENGWSPTPRCIRVKTCSKSSIDIENGFISES
QYTYALKEKAKYQCKLGYVTADGETSGSITCGKDGWSAQPTCIKSCDIPVFMNARTK
NDFTWFKLNDTLDYECHDGYESNTGSTTGSIVCGYNGWSDLPICYERECELPKIDVHL
VPDRKKDQYKVGEVLKFSCKPGFTIVGPNSVQCYHFGLSPDLPICKEQVQSCGPPPELL
NGNVKEKTKEEYGHSEVVEYYCNPRFLMKGPNKIQCVDGEWTTLPVCIVEESTCGDIP
ELEHGWAQLSSPPYYYGDSVEFNCSESFTMIGHRSITCIHGVWTQLPQCVAIDKLKKC
KSSNLIILEEHLKNKKEFDHNSNIRYRCRGKEGWIHTVCINGRWDPEVNCSMAQIQLCP
PPPQIPNSHNMTTTLNYRDGEKVSVLCQENYLIQEGEEITCKDGRWQSIPLCVEKIPCS
QPPQIEHGTINSSRSSQESYAHGTKLSYTCEGGFRISEENETTCYMGKWSSPPQCEGLPC
KSPPEISHGVVAHMSDSYQYGEEVTYKCFEGFGIDGPAIAKCLGEKWSHPPSCIKTDCL
SLPSFENAIPMGEKKDVYKAGEQVTYTCATYYKMDGASNVTCINSRWTGRPTCRDTS
CVNPPTVQNAYIVSRQMSKYPSGERVRYQCRSPYEMFGDEEVMCLNGNWTEPPQCK
DSTGKCGPPPPIDNGDITSFPLSVYAPASSVEYQCQNLYQLEGNKRITCRNGQWSEPPK
CLHPCVISREIMENYNIALRWTAKQKLYSRTGESVEFVCKRGYRLSSRSHTLRTTCWD
GKLEYPTCAKR
(SEQ ID NO: 38)
MRLLAKIICLMLWAICVAEDCNELPPRRNTEILTGSWSDQTYPEGTQAIYKCRPGYRSL
GNVIMVCRKGEWVALNPLRKCQKRPCGHPGDTPFGTFTLTGGNVFEYGVKAVYTCN
EGYQLLGEINYRECDTDGWTNDIPICEVVKCLPVTAPENGKIVSSAMEPDREYHFGQA
VRFVCNSGYKIEGDEEMHCSDDGFWSKEKPKCVEISCKSPDVINGSPISQKIIYKENERF
QYKCNMGYEYSERGDAVCTESGWRPLPSCEEKSCDNPYIPNGDYSPLRIKHRTGDEIT
YQCRNGFYPATRGNTAKCTSTGWIPAPRCTLKPCDYPDIKHGGLYHENMRRPYFPVA
VGKYYSYYCDEHFETPSGSYWDHIHCTQDGWSPAVPCLRKCYFPYLENGYNQNHGR
KFVQGKSIDVACHPGYALPKAQTTVTCMENGWSPTPRCIRVKTCSKSSIDIENGFISES
QYTYALKEKAKYQCKLGYVTADGETSGSITCGKDGWSAQPTCIKSCDIPVFMNARTK
NDFTWFKLNDTLDYECHDGYESNTGSTTGSIVCGYNGWSDLPICYERECELPKIDVHL
VPDRKKDQYKVGEVLKFSCKPGFTIVGPNSVQCYHFGLSPDLPICKEQVQSCGPPPELL
NGNVKEKTKEEYGHSEVVEYYCNPRFLMKGPNKIQCVDGEWTTLPVCIVEESTCGDIP
ELEHGWAQLSSPPYYYGDSVEFNCSESFTMIGHRSITCIHGVWTQLPQCVAIDKLKKC
KSSNLIILEEHLKNKKEFDHNSNIRYRCRGKEGWIHTVCINGRWDPEVNCSMAQIQLCP
PPPQIPNSHNMTTTLNYRDGEKVSVLCQENYLIQEGEEITCKDGRWQSIPLCVEKIPCS
QPPQIEHGTINSSRSSQESYAHGTKLSYTCEGGFRISEENETTCYMGKWSSPPQCEGLPC
KSPPEISHGVVAHMSDSYQYGEEVTYKCFEGFGIDGPAIAKCLGEKWSHPPSCIKTDCL
SLPSFENAIPMGEKKDVYKAGEQVTYTCATYYKMDGASNVTCINSRWTGRPTCRDTS
CVNPPTVQNAYIVSRQMSKYPSGERVRYQCRSPYEMFGDEEVMCLNGNWTEPPQCK
DSTGKCGPPPPIDNGDITSFPLSVYAPASSVEYQCQNLYQLEGNKRITCRNGQWSEPPK
CLHPCVISREIMENYNIALRWTAKQKLYSRTGESVEFVCKRGYRLSSRSHTLRTTCWD
GKLEYPTCAKR.

The invention provides embodiments in which a polypeptide of SEQ ID NO: 37 is administered. The invention provides embodiments in which a polypeptide of SEQ ID NO: 38 is administered. The invention provides embodiments in which a polypeptide at least 90% identical to SEQ ID NO: 37 and having Y at position corresponding to 402 is administered. The invention provides embodiments in which a polypeptide at least 90% identical to SEQ ID NO: 37 and not having Y at position corresponding to 402 is administered. The invention provides embodiments in which a polypeptide at least 90% identical SEQ ID NO: 38 and having H at position corresponding to position 402 is administered. In some embodiments a composition in which at least 50%, 60%, 70%, 80%, 90%, 95%, 98%, 99% or 100% of the CFH polypeptide therein has Y at position 402 (“CFH Y402”) is administered. In some embodiments a composition in which at least 50%, 60%, 70%, 80%, 90%, 95%, 98%, 99% or 100% of the CFH polypeptide therein has H at position 402 (“CFH H402”) is administered. In some embodiments a composition in which at least 60%, 70%, 80%, 90%, 95%, 98%, 99% or 100% of the CFH polypeptide therein does not have Y at position 402 is administered.

In certain embodiments of the invention the CFH polypeptide or modulator is first administered within 1, 2, 4, 6, 8, 12, or 24 hours following injury. In some embodiments the CFH polypeptide or modulator is first administered at least 5 minutes following injury but within the first 30 minutes following injury, or in some embodiments within the first 1 hour following injury. In some embodiments the CFH polypeptide or modulator is first administered at least 5 minutes after injury but within 2, 3, 4, 6, 8, 12, 24, 48, 72, or 96 hours after injury. In some embodiments the CFH polypeptide or modulator is administered multiple times within any of the afore-mentioned time intervals. In some embodiments the CFH polypeptide is administered at least once at least 24, at least 48, or at least 72 hours following injury, up to about 2 weeks following injury, and optionally at one or more subsequent time points.

Typically, CFH polypeptide or modulator is administered intravenously although other routes of administration may be used in certain embodiments of the invention. In some embodiments of the invention the CFH polypeptide or modulator is administered as an intravenous (IV) bolus. In some embodiments the CFH polypeptide or modulator is administered by IV infusion. In some embodiments of the invention the CFH polypeptide or modulator is administered as an IV bolus followed by an IV infusion. In some embodiments, the method comprises administering the CFH polypeptide or modulator as an IV drip. An early step in treating a trauma victim is often placing an IV line and providing IV fluids. In some embodiments the CFH polypeptide or modulator is added to such IV fluid. The CFH polypeptide may be administered in an amount sufficient to achieve a concentration or amount of CFH polypeptide in the subject's blood to within a normal range for CHF, e.g., if the subject has experienced significant blood loss. In some embodiments the CFH polypeptide is administered in an amount sufficient to achieve a concentration or amount of CFH polypeptide in the subject's blood to between 1 and 2, or between 2 and 5, times the average value for CHF in the blood of normal individuals. In some embodiments the CFH polypeptide is administered in an amount sufficient to achieve a concentration or amount of CFH polypeptide in the subject's blood to between 1 and 2, or between 2 and 5, times the upper limit of normal range for CHF in the blood of normal individuals.

Pharmaceutical compositions comprising a CFH polypeptide suitable for use in a trauma patient are provided. In some embodiments at least 50%, 60%, 70%, 80%, 90%, 95%, 98%, 99% or 100% of the CFH polypeptide therein has H at position 402. In some embodiments between 95% and 100%, e.g., 100% of the CFH polypeptide therein has H at position 402. The invention provides a composition that comprises a CFH polypeptide and adrenomedullin peptide, wherein at least 50%, 60%, 70%, 80%, 90%, 95%, 98%, 99% or 100% of the CFH polypeptide therein has H at position 402. In some embodiments between 95% and 100%, e.g., 100% of the CFH polypeptide therein has H at position 402. In some embodiments of the invention the CFH polypeptide is in a complex with adrenomedullin peptide. In some embodiments of the invention a composition comprising CFH polypeptide wherein at least 50%, 60%, 70%, 80%, 90%, 95%, 98%, 99% or 100% of the CFH polypeptide therein has H at position 402 is administered in combination with adrenomedullin. In some embodiments of the invention a CFH polypeptide is administered in a composition that contains substantially less adrenomedullin peptide than CFH polypeptide (e.g., the concentration of adrenomedullin is no more than 1%, no more than 5%, no more than 10%, or no more than 25% of the concentration of CFH polypeptide on a molar basis. In some embodiments of the invention no more than 1%, no more than 5%, no more than 10%, or no more than 25% of the CFH polypeptide administered is in a complex with adrenomedullin peptide. In some embodiments the composition is essentially free of adrenomedullin, e.g., it comprises recombinantly produced CFH produced by cells that do not express adrenomedullin, and no adrenomedullin has been added. In some embodiments of the invention a CFH polypeptide is not administered in combination with adrenomedullin.

Methods for Identifying Agents for Use to Treat Trauma

The invention provides a method of identifying an agent for use in treating a trauma patient in the immediate post-trauma period, the method comprising: (a) contacting an agent with a complement component, wherein the agent binds to the complement component to form an agent-component complex, and wherein the complement component is inactive when present in the complex; and (b) determining whether the complement component is significantly recoverable from the complex in active form, wherein if the complement component is significantly recoverable from the complex in active form, the agent is useful for treatment of a trauma patient in the immediate post-trauma period. In some embodiments of the invention the contacting step is performed in vitro. In some embodiments of the method the contacting step is performed in vivo. In some embodiments of the method the agent inhibits activation of the complement component. “Significantly recoverable in active form” means that at least 50%, e.g., at least 75%, e.g., at least 90% of the complement component dissociates from the complex and retains activity when the complex is incubated (e.g., for between 1 and 24 hours) under conditions consistent with maintaining activatability of the complement component.

The invention also provides a method of identifying an agent for use in treating a trauma patient in the immediate post-trauma period at a given dose, the method comprising: (a) administering the complement activation inhibitor to a mammalian subject at the given dose; (b) obtaining a measurement of the degree of complement inhibition in a sample obtained from the subject at a first time point, wherein the first time point is within 2 hours following administration of the complement activation inhibitor; (c) obtaining a measurement of the degree of complement inhibition in a sample obtained from the subject at a second time point, wherein the second time point is between 24 and 72 hours following administration of the complement activation inhibitor; and (d) comparing the degree of complement inhibition in the samples obtained at the first and second time points, wherein the complement activation inhibitor is identified as useful for treating a trauma patient in the immediate post-trauma period at the given dose if the degree of complement inhibition has decreased by at least 75% between the first and second time points. In some embodiments of the method the complement activation inhibitor is identified as useful for treating a trauma patient in the immediate post-trauma period at the given dose if the degree of complement inhibition has decreased by at least 90% between the first and second time points. In some embodiments of the method the degree of complement inhibition at first and second time points is obtained by comparing potential complement activity at the first or second time point with potential complement activity at a third time point when the subject has not received a complement inhibitor within the preceding 6 weeks. In some embodiments of the method the third time point is a time point at which the subject is in an essentially normal physiological state. In some embodiments of the method the third time point is a time point at which the subject has experienced traumatic injury within the preceding 1 hour.

The invention provides a method of identifying a complement activation inhibitor as useful for treating a trauma patient in the immediate post-trauma period at a given dose comprising: (a) administering the complement activation inhibitor to a mammalian subject at the given dose; (b) obtaining a measurement of the degree of potential complement activity in a sample obtained the subject at a first time point, wherein the first time point is between 24 and 72 hours following administration of the complement inhibitor; and (c) comparing the degree of potential complement activity in a sample obtained from the subject at the first time point with the degree of potential complement activity in a sample obtained from the subject at a second time point at which the subject is in an essentially normal physiological condition, wherein the complement inhibitor is identified as useful for treating a trauma patient in the immediate post-trauma period at the given dose if the degree of potential complement activity at the first time point is at least 75% as great as the degree of potential complement activity at the second time point. In some embodiments of the method the complement activation inhibitor is identified as useful for treating a trauma patient in the immediate post-trauma period at the given dose if the degree of potential complement activity at the first time point is at least 90% as great as the degree of potential complement activity at the second time point.

The invention also provides a method of identifying a complement activation inhibitor useful for treating a trauma patient in the immediate post-trauma period at a given dose, the method comprising: (a) administering the complement activation inhibitor to a mammalian subject at the given dose at a first time point, wherein the subject has experienced traumatic injury within the 6 hours preceding the first time point; (b) determining whether the complement activation inhibitor at least partly preserves potential complement activity at a second time point when administered at the given dose, wherein the second time point is between 24 and 96 hours post-injury, and wherein the complement inhibitor is considered to at least partly preserve potential complement activity when administered at the given dose if the degree of potential complement activity at the second time point is greater than a suitable control value and (c) identifying the complement activation inhibitor as useful for treating a trauma patient if the complement activation inhibitor at least partly preserves potential complement activity. In some embodiments of the method the subject has experienced traumatic injury within the 1 hour preceding the first time point. In some embodiments of the method the second time point is at least 48 hours post-injury. The control value may be the value that would be expected had the complement activation inhibitor not been administered, which may be determined experimentally or based on historical data (e.g., in subjects suffering trauma of similar severity) or predicted based on measurements made at the first time point.

Formulations, Kits, and Articles for Trauma Therapy

The invention encompasses administration of a complement inhibitor as described herein, optionally in conjunction with additional therapy suitable for a trauma patient. Such therapy may include administration of any active agent known in the art for treating trauma patients, such as blood products, recombinant factor VIIa or other clotting factors, blood substitutes, fluids, volume expanders, anti-hypotensive agents, antibiotics, etc. Also of use are topical agents effective in controlling hemorrhage such as chitosan wafer dressing (HemCon [HC]), zeolite powder dressing (QuikClot [QC]), and chitosan granule dressing (CELOX [CX]). The invention provides fixed dose formulations containing sufficient complement inhibitor to significantly inhibit complement activation in a child or adult human following a single intravenous (IV) or oral administration. Such formulations may take the form of a physically discrete unit that contains a predetermined quantity of complement inhibitor. For example, such formulations could reduce systemic complement activity by between 50% and 99%, e.g., by at least 50%, 75%, or 90%, relative to levels present prior to administration or relative to normal or average levels. The formulation may be a liquid formulation provided in a vial, a prefilled syringe, etc. One aspect of the invention is articles of manufacture containing a fixed dose of complement inhibitor in a convenient form for rapid administration, e.g., rapid IV administration, to a subject. In certain embodiments the complement inhibitor may be added directly to an IV fluid solution. The invention provides fixed dose formulations containing an appropriate amount of complement inhibitor to be added directly to a standard IV solution bag, wherein administration of the fluid in such bag would reduce systemic complement activity by between 50% and 99%, e.g., by at least 50%, 75%, or 90%, relative to levels present prior to administration or relative to normal or average levels.

The invention provides a kit containing at least one fixed dose formulation comprising a complement inhibitor. Also provided are kits containing multiple fixed dose formulations of a complement inhibitor, wherein at least two of the fixed dose formulations contain different amounts of the complement inhibitor, wherein such different amounts are selected to achieve a desired amount of complement inhibition in, e.g., infants, children, and adults. In some embodiments fixed dose formulations containing three different amounts of a complement inhibitor are included. Emergency response vehicles, e.g., ambulances, may be equipped with such kits.

The invention provides IV solution containers comprising an IV solution and complement inhibitor as described herein. In some embodiments the IV solution container has a distinct color, marking, or design as compared with conventional IV solution containers. For example, the container may be red, may be marked with a particular legend, etc. The container may be a standard IV solution bag such as a 500 ml flexible plastic IV solution bag. The fluid contained in such IV solution container could be any fluid suitable for administration to a trauma patient. Examples include physiologically acceptable saline (e.g., 0.9% saline), 5% dextrose in water, Ringer's lactate, etc. The solution could contain a colloid or crystalloid useful for volume expansion. Examples of colloids include hydroxyethyl starch (including various forms and sizes thereof) (sold under names such as Hetastarch®; HAES-steril®, Voluven®, etc.)

A kit can comprise or be provided together with instructions for use of the composition contained in the kit for treating an individual who has suffered a traumatic injury. For example, if the composition comprises a complement activation inhibitor, such instructions may indicate that the composition should be administered within 24 hours after occurrence of the injury. If the composition comprises a complement component or an inhibitor of an endogenous complement inhibitor, the instructions may indicate that the composition should be administered within 4 hours and 4 days after occurrence of the injury.

Pharmaceutical Compositions

Suitable preparations, e.g., substantially pure preparations of the complement activation inhibitor, the complement component, and/or the complex comprising a complement component and a stabilizing agent, may be combined with pharmaceutically acceptable carriers, diluents, solvents, etc., to produce an appropriate pharmaceutical composition. In some embodiments the composition comprises a compstatin analog. In some embodiments the composition comprises a CFH polypeptide.

In various embodiments of the invention an effective amount of the pharmaceutical composition is administered to a subject by any suitable route of administration. In some embodiments the composition is administered intravenously. In some embodiments the composition is administered intracranially or intrathecally, e.g., if the individual has suffered head injury or injury to the spinal column or spinal cord. In some embodiments the composition is administered locally, e.g., at a site of tissue damage. A composition may be administered once or more than once within a given time interval. In some embodiments the blood or plasma level of a complement activation inhibitor, complement component, split product, or complement activation capacity is monitored, and the dose or dosing interval is selected or adjusted to achieve a desired level. A pharmaceutical composition can be provided together with instructions for use of the composition to treat an individual who has suffered trauma. The instructions may describe amounts to be administered, methods of reconstituting a powder formulation, routes of administration, time intervals, indications, etc.

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices when administered appropriately are often preferred. Data obtained from cell culture assays and animal studies can be used in formulating a range of dosage for use in humans. In some embodiments, dosage of such compounds lies within a range of circulating concentrations that include the ED₅₀ with little or no toxicity. The dosage can vary within this range depending upon the dosage form employed and the route of administration utilized. In vitro assays can be used to determine certain parameters, e.g., ability to inhibit complement activation, etc. Compositions and doses can be tested in animal models, if desired, to obtain information that might predict useful doses in humans. Levels in plasma can be measured, for example, by high performance liquid chromatography or other suitable methods depending on the compound. Animal models relevant to traumatic injury and potential poor outcomes are known in the art. Animal models include contusion models, ischemia-reperfusion injury models, wound models, spinal cord injury models, shock models, burn models, inhalation injury models, sepsis models, hemorrhage or blood loss models, Different doses and dosing regimens may be tested in clinical trials. For example, people suffering severe trauma (e.g., car crash victims) can be randomized to either receive or not receive an inventive complement activation inhibitor and/or pro-repletion therapy and the outcomes compared.

In some embodiments a dose ranging from about 0.001 mg/kg to 100 mg/kg body weight, e.g., about 0.01 to 25 mg/kg body weight, e.g., about 0.1 to 20 mg/kg body weight, e.g., about 1 to 10 mg/kg of a compound described herein is administered. A pharmaceutical composition can be administered at various intervals and over different periods of time as required. The skilled artisan will appreciate that certain factors can influence the dosage and timing required to effectively treat a subject, including but not limited to the severity of the trauma, other treatments being administered, the general health and/or age of the subject, and diseases that may be present.

The term “pharmaceutically acceptable carrier or vehicle” refers to a non-toxic carrier or vehicle that does not destroy the pharmacological activity of the compound with which it is formulated. Pharmaceutically acceptable carriers or vehicles that may be used in the compositions of this invention include, but are not limited to, water, physiological saline, 5% dextrose, and the like. The composition may include other components as appropriate for the formulation desired, e.g., buffer substances, anti-bacterial agents, etc. Supplementary active compounds, e.g., compounds independently useful for treating a trauma or surgery patient, can also be incorporated into the compositions.

It will be appreciated that the complement activation inhibitor and/or additional or other active agent(s) can be provided as a pharmaceutically acceptable salt. Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Solutions or suspensions used for parenteral (e.g., intravenous) or intramuscular, administration can include the following components in various embodiments of the invention: a sterile diluent such as water for injection, saline solution, antibacterial agents such as benzyl alcohol or methyl parabens; antioxidants such as ascorbic acid or sodium bisulfite, chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. Sterile injectable solutions can be prepared by incorporating the complement inhibitor in the required amount in an appropriate solvent with one or a combination of ingredients such as buffers, antibacterial agents, etc., as required, followed by filtered sterilization. Preferably solutions for injection are free of endotoxin. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation include vacuum drying and freeze-drying which yields a powder of the active ingredient plus any additional desired ingredient from a previously sterile-filtered solution thereof.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. For example, and without limitation, it is understood that where claims or description indicate that a residue at a particular position may be selected from a particular group of amino acids or amino acid analogs, the invention includes individual embodiments in which the residue at that position is any of the listed amino acids or amino acid analogs. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. In particular, any claim that is dependent on another claim can be modified to include one or more elements or limitations found in any other claim that is dependent on the same base claim. Furthermore, where the claims recite a composition, it is to be understood that methods of administering the composition according to any of the methods disclosed herein, and methods of using the composition for any of the purposes disclosed herein are included, and methods of making the composition according to any of the methods of making disclosed herein are included, unless otherwise indicated or unless it would be evident to one of ordinary skill in the art that a contradiction or inconsistency would arise.

Where elements are presented as lists, e.g., in Markush group format, each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

It should be understood that measurements relating to amount or concentration of an entity (e.g., a complement component, complement inhibitor, etc.) “in the blood” or assessments of complement activation capacity “in the blood” discussed herein are often performed on plasma or serum. Where compositions or methods of the invention mention or involve such measurement or assessment and refer to “blood”, it should be understood that embodiments in which such measurement or assessment is made on blood, plasma, or serum are provided by the instant invention.

As discussed above, certain of the inventive methods comprise administering a composition or agent to an individual within a time interval of interest relative to the time at which the individual has suffered a traumatic injury, e.g., within 24 hours after occurrence of the injury or between 4 hours and 4 days after occurrence of the injury. The invention encompasses embodiments of these methods in which the composition or agent is administered within the time interval and is not administered thereafter within a period of time after the time interval. The period of time after the time interval may be, e.g., 10 days, 2 weeks, 4 weeks, 6 weeks, the period of time in which the individual remains at risk of poor outcome, or the period of time during which the individual remains in an intensive care unit or remains hospitalized. The invention also encompasses embodiments in which the composition or agent is administered within the time interval and is also administered thereafter within a period of time after the time interval (once or more than once in various embodiments).

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where phrases such as “less than X” or “greater than Y” are used (with X and Y being numbers or percentages), it should be understood that “less than X but greater than X1” and “greater than Y but less than Y1”, respectively, are disclosed, wherein X1 is any value less than X, and Y1 is any value greater than Y, provided in either case that such value is reasonable in the context. Where the phrase “at least X” is used, “at least X and no more than X1” is disclosed, where X1 can be any value greater than X, provided such value is reasonable in the context. In addition, any particular embodiment, aspect, element, feature, etc., of the present invention, e.g., any particular complement activation inhibitor or class of complement activation inhibitors, may be explicitly excluded from the claims.

1. A method of treating an individual who has suffered a traumatic injury comprising administering a complement activation inhibitor to the individual within 24 hours after the injury, wherein the complement inhibitor (i) acts at or above the level of C3 activation; and (ii) does not cause significant depletion or irreversible inactivation of an unactivated complement component.

2. The method of claim 1, wherein the complement activation inhibitor inhibits depletion of C3.

3. The method of claim 1, wherein the complement activation inhibitor acts on a complement component selected from the group consisting of: C1, C2, C3, C4, factor B, factor D, or an active fragment of one or more of the foregoing.

4. (canceled)

5. The method of claim 1, wherein the complement inhibitor has an average half-life of 24 hours or less in humans under normal conditions.

6.-19. (canceled)

20. The method of claim 1, wherein the complement inhibitor inhibits a C3 convertase.

21. The method of claim 1, wherein the complement inhibitor binds to C3 and inhibits its activation.

22. The method of claim 1, wherein the complement inhibitor comprises a compstatin analog.

23. The method of claim 1, wherein the complement activation inhibitor comprises a compstatin analog having a sequence selected from the group consisting of: SEQ ID NOs: 14, 21, 28, 29, 30, 32, 33, 34, or 36.

24. The method of claim 1, wherein the complement activation inhibitor comprises a VCCP or VCIP or complement inhibiting altered version thereof.

25. The method of claim 1, wherein the complement activation inhibitor comprises a mammalian complement regulatory protein or a complement inhibiting altered version thereof.

26. The method of claim 1, wherein the complement activation inhibitor comprises a mammalian complement control protein or a complement inhibiting fragment or altered version thereof, wherein the mammalian complement control protein is complement factor H(CFH), decay accelerating factor (DAF), or membrane cofactor protein (MCP).

27.-37. (canceled)

38. The method of claim 1, further comprising administering to the individual an unactivated complement component.

39.-40. (canceled)

41. The method of claim 38, wherein the complement component is administered at least 24 hours after the injury.

42.-43. (canceled)

44. The method of claim 38, wherein the complement component is an at least partially purified or recombinantly produced protein.

45.-52. (canceled)

53. The method of claim 1, comprising administering a complement activation inhibitor to the individual in the immediate post-trauma period in an amount effective to reduce trauma-induced complement depletion by at least 50%, wherein the complement inhibitor does not itself cause significant depletion or irreversible inactivation of an unactivated complement component, and wherein the complement inhibitor is administered in an amount such that at least 90% of the complement inhibitor administered is gone from the blood within 1 week after the injury.

54.-135. (canceled)


SEQUENCE LISTING (44 sequences)

SEQ ID NO: 1 - protein, 9 aa, Artificial Sequence
  Synthetic (sequence used for exemplary purposes to define “identity”)
AKLXXXSIG

SEQ ID NO: 2 - protein, 6 aa, Artificial Sequence
  Synthetic
XQDXGX

SEQ ID NO: 3 - protein, 5 aa, Artificial Sequence
  Synthetic; core sequence of peptide
XQDXG

SEQ ID NO: 4 - protein, 6 aa, Artificial Sequence
  Synthetic
XQDXGX

SEQ ID NO: 5 - protein, 13 aa, Artificial Sequence
  Synthetic
XXXXQDXGXXXXX

SEQ ID NO: 6 - protein, 13 aa, Artificial Sequence
  Synthetic
XCVXQDXGXHRCX

SEQ ID NO: 7 - protein, 13 aa, Artificial Sequence
  Synthetic
XCVXQDXGXHRCX

SEQ ID NO: 8 - protein, 13 aa, Artificial Sequence
  Synthetic; compstatin
ICVVQDWGHHRCT

SEQ ID NO: 9 - protein, 13 aa, Artificial Sequence
  Synethetic; compstatin analog
ICVVQDWGHHRCT

SEQ ID NO: 10 - protein, 13 aa, Artificial Sequence
  Synthetic; compstatin analog
ICVYQDWGAHRCT

SEQ ID NO: 11 - protein, 13 aa, Artificial Sequence
  Synthetic; compstatin analog
ICVWQDWGAHRCT

SEQ ID NO: 12 - protein, 13 aa, Artificial Sequence
  Synthetic; compstatin analog
ICVWQDWGAHRCT

SEQ ID NO: 13 - protein, 13 aa, Artificial Sequence
  Synthetic; compstatin analog
ICVWQDWGAHRCT

SEQ ID NO: 14 - protein, 13 aa, Artificial Sequence
  Synthetic; compstatin analog
ICVAQDWGAHRCT

SEQ ID NO: 15 - protein, 13 aa, Artificial Sequence
  Synthetic; compstatin analog
ICVAQDWGAHRCT

SEQ ID NO: 16 - protein, 13 aa, Artificial Sequence
  Synthetic; compstatin analog
ICVAQDWGAHRCT

SEQ ID NO: 17 - protein, 13 aa, Artificial Sequence
  Synthetic; compstatin analog
ICVGQDWGAHRCT

SEQ ID NO: 18 - protein, 13 aa, Artificial Sequence
  Synthetic; compstatin analog
ICVGQDWGAHRCT

SEQ ID NO: 19 - protein, 13 aa, Artificial Sequence
  Synthetic; compstatin analog
ICVWQDWGAHRCT

SEQ ID NO: 20 - protein, 13 aa, Artificial Sequence
  Synthetic; compstatin analog
ICVAQDWGAHRCT

SEQ ID NO: 21 - protein, 13 aa, Artificial Sequence
  Synthetic; compstatin analog
ICVAQDWGAHRCT

SEQ ID NO: 22 - protein, 13 aa, Artificial Sequence
  Synthetic; compstatin analog
ICVAQDWGAHRCT

SEQ ID NO: 23 - protein, 13 aa, Artificial Sequence
  Synthetic; compstatin analog
ICVAQDWGAHRCT

SEQ ID NO: 24 - protein, 13 aa, Artificial Sequence
  Synthetic; compstatin analog
ICVWQDWGXHRCT

SEQ ID NO: 25 - protein, 16 aa, Artificial Sequence
  Synthetic; compstatin analog
GICVWQDWGAHRCTAN

SEQ ID NO: 26 - protein, 13 aa, Artificial Sequence
  Synthetic; compstatin analog
ICVWQDWGAHRCT

SEQ ID NO: 27 - protein, 13 aa, Artificial Sequence
  Synthetic; compstatin analog
ICVWQDWGAHRCT

SEQ ID NO: 28 - protein, 13 aa, Artificial Sequence
  Synthetic; compstatin analog
ICVWQDWGAHRCT

SEQ ID NO: 29 - protein, 13 aa, Artificial Sequence
  Synthetic; compstatin analog
ICVWQDWGAHRCT

SEQ ID NO: 30 - protein, 13 aa, Artificial Sequence
  Synthetic; compstatin analog
ICVWQDWGAHRCT

SEQ ID NO: 31 - protein, 13 aa, Artificial Sequence
  Synthetic; compstatin analog
ICVWQDWGAHRCT

SEQ ID NO: 32 - protein, 13 aa, Artificial Sequence
  Synthetic; compstatin analog
ICVWQDWGAHRCT

SEQ ID NO: 33 - protein, 15 aa, Artificial Sequence
  Synthetic; compstatin analog
GICVWQDWGAHRCTN

SEQ ID NO: 34 - protein, 13 aa, Artificial Sequence
  Synthetic; compstatin analog
ICVWQDWGAHRCT

SEQ ID NO: 35 - protein, 13 aa, Artificial Sequence
  Synthetic; compstatin analog
ICVWQDWGAHRCT

SEQ ID NO: 36 - protein, 15 aa, Artificial Sequence
  Synthetic; compstatin analog
GICVWQDWGAHRCTN

SEQ ID NO: 37 - protein, 1231 aa, Homo sapiens
  misc_feature (1)..(1231): Complement Factor H polypeptide
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 38 - protein, 1231 aa, Homo sapiens
  misc_feature (1)..(1231): Complement Factor H polypeptide
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 39 - protein, 5 aa, Artificial Sequence
  Synthetic peptide
XPXWR

SEQ ID NO: 40 - protein, 6 aa, Artificial Sequence
  Synthetic peptide
XXPXWR

SEQ ID NO: 41 - protein, 5 aa, Artificial Sequence
  Synthetic peptide
XPXWR

SEQ ID NO: 42 - protein, 5 aa, Artificial Sequence
  Synthetic peptide
XPXWR

SEQ ID NO: 43 - protein, 6 aa, Artificial Sequence
  Synthetic peptide
FXPXWR

SEQ ID NO: 44 - protein, 6 aa, Artificial Sequence
  Synthetic peptide
FKPXWR